(19) United States
(12) Patent Application Publication
(10) Pub. No.: US 2023/0241101 A1
(43) Pub. Date: Aug. 3, 2023

(54) THERAPEUTIC COMPOSITION AND METHOD FOR TREATING CORONAVIRUS INFECTION

(71) Applicant: BEIJING BJUT SCIENCE AND TECHNOLOGY PARK CO., LTD, Beijing (CN)

(72) Inventor: Xueyun GAO, Beijing (CN)

(73) Assignee: BEIJING BJUT SCIENCE AND TECHNOLOGY PARK CO., LTD, Beijing (CN)

(21) Appl. No.: 17/927,390

(22) PCT Filed: May 20, 2021
(86) PCT No.: PCT/CN2021/094868

§ 371 Date: Nov. 23, 2022

Related U.S. Application Data
Application No. PCT/CN2021/094868 is a continuation of application No. 17/181,597, filed on Feb. 22, 2021.
(60) Provisional application No. 63/030,526, filed on May 27, 2020.
(60) Provisional application No. 63/045,128, filed on Jun. 28, 2020.

(30) Foreign Application Priority Data
Jan. 27, 2021 (CN) 202110109926.4

(51) Int. Cl. A61K 33/242; A61K 31/80; A61P 31/14; A61K 47/64; A61K 31/7135

(52) U.S. Cl. CPC A61K 33/242; A61K 31/80; A61K 31/7135; A61K 47/645; A61P 31/14

(57) ABSTRACT

Provided is a therapeutic composition for treatment of coronavirus infection. The therapeutic composition contains a gold compound that generates Au—S bonds at the active pockets of the main protease of the virus while suppressing the virus-induced inflammations in the body.

### CROSS-REFERENCE TO RELATED APPLICATIONS

This application is a U.S. National Phase Application under 35 U.S.C. 371 of International Application No. PCT/CN2021/094868, filed on May 20, 2021, The entire disclosure of the above application is expressly incorporated by reference herein. Priority is claimed to the U.S. Provisional Pat. Application No. 63/030,526, filed on May 27, 2020, entitled “Molecules inhibit the enzyme activity of 3-chymotrypsin-like cysteine protease of SARS-CoV-2 virus,” the entirety of which is hereby incorporated by reference for all purposes. Priority is also claimed to the U.S. Provisional Pat. Application No. 63/045,128, filed on Jun. 28, 2020, entitled “Au ions bind the Cys145 and Cys156 residue of the 3-chymotrypsin-like cysteine protease of SARS-CoV-2 and inhibit its activity”, the entirety of which is hereby incorporated by reference for all purposes. Priority is also claimed to the U.S. Pat. Application No. 17/181,597, filed on Feb. 22, 2021, entitled “Therapeutic Agents for Treatment of Coronavirus Infection,” the entirety of which is hereby incorporated by reference for all purposes. Priority is also claimed to the China Patent Application No. 202110109926.4, filed on Jan. 27, 2021, entitled “The use of gold complexes in the manufacture of drugs for preventing coronavirus disease 2019”, the entirety of which is hereby incorporated by reference for all purposes.

### REFERENCE TO SEQUENCE LISTING

A computer readable XML file entitled “GWPCTP20220801328_Sequence listing.xml”, that was created on Oct. 21, 2022, with a file size of about 7,461 bytes, contains the sequence listing for this application, has been filed with this application, and is hereby incorporated by reference in its entirety.

### TECHNICAL FIELD

The present application relates to the treatment of coronavirus SARS-CoV-2 infection and more particularly to a gold compound that inhibits the replication of SARS-CoV-2 while suppressing the cytokine storm syndromes.

### BACKGROUND

Note that the points discussed below may reflect the hindsight gained from the disclosed inventions, and are not necessarily admitted to be prior art.

Coronavirus diseases, especially COVID-19, are infectious diseases that have spread worldwide and have caused great damage to the economies of the world. It is commonly recognized that severe acute respiratory syndrome coronavirus 2 (SARS-CoV-2) is the cause of COVID-19. Symptoms of COVID-19 include high fever, cough, trouble breathing, persistent pain or pressure in the chest, new confusion, inability to wake or stay awake, bluish lips or face, and organ failure. It is believed that many of the symptoms are caused by cytokine storm phenomena during which multiple cytokines, such as TNF-α, IL-1, IL-6, IL-12, IFN-α, IFN-β, IFN-γ, MCP-1, and IL-8, are rapidly and massively produced in the body fluid when the body’s immune system is over-activated by the virus infection. Cytokine storm has been an important cause of acute respiratory distress syndrome and multiple organ failures. Once a cytokine storm occurs, it can quickly cause single or multiple organ failure and becomes eventually life-threatening.

Many studies have been conducted in searching the cures for COVID-19. Current drug design strategies focus either on identifying antibodies or chemical drugs that can either inhibit virus replication or suppress inflammatory cytokines. Generally, chemical drugs are of unique advantages in dealing with the COVID-19 pandemic: chemical drugs can be easily produced on a large scale at a low cost and thus can satisfy the treatment demands for a large number of COVID-19 patients in a short time. More importantly, chemical drugs allow for efficient handling, storage, and distribution to patients living in environments unsuitable for biological agents. However, despite the tremendous global efforts in identifying suitable chemical drugs for the treatment of COVID-19, no chemical drug has been demonstrated to be able to treat COVID-19 infections effectively. For example, the FDA-approved drug REMDESIVIR was shown to only inhibit SARS-CoV-2 replication but failed to protect lungs from inflammation injury in patients with severe infections. On the other hand, RUXOLITINIB and ACALABRUTINIB were shown to suppress inflammatory cytokine spikes but failed to inhibit virus replication.

It would be more effective and desirable if an active ingredient chemical compound of a medicament can both inhibit virus replication and suppress cytokine storms. In this application, such novel therapeutic agents for potential treatment of COVID-19 are identified.

### SUMMARY

Gold compounds have been used for the treatment of rheumatoid arthritis (RA) through both oral route and parenteral route. Among them, AURANOFIN (AF) and aurothioglucose (also known as gold thioglucose), sodium aurothiomalate (gold sodium thiomalate) are FDA-approved drugs, all inhibiting inflammatory cytokines associated with rheumatoid arthritis, but they have side effects that can be severe for some patients. Recently, the present inventor in U.S. Pat. No. 9,993,562 (it entirety is hereby incorporated by reference) demonstrated that gold cluster compounds were able to achieve therapeutic outcomes that are comparable to AURANOFIN (AF), but were without some of AURANOFIN’s side effects.

The present application further shows that gold compounds for rheumatoid arthritis treatment can also counter lung inflammations in COVID-19 animals. In addition, the gold compounds AURANOFIN (AF) and gold cluster GA are shown to form Au-S bonds in the active pockets of the viral cysteine protease M^{pro}. Because the viral 3-chymotrypsin-like cysteine protease (3CLpro, also called M^{pro}) enzyme controls the coronavirus replication and it is essential for its life cycle, M^{pro} has been a drug discovery target in seeking a treatment for COVID-19. In GA-treated COVID-19 mice and rats, not only were the replications of SARS-CoV-2 significantly reduced, but also were the inflammatory cytokines of IL-6, IL-1β, and TNF-α suppressed, and no significant side effects were observed even at relatively high doses.

One aspect of the invention relates to a therapeutic composition for treatment of coronavirus infection in an animal, comprising: a gold compound stored in a liquid form as an active ingredient wherein a gold atom forms an Au—S bond in the coronavirus’ protease active pocket.

In one embodiment of the invention, the gold compound is AURANOFIN having molecule formula C₂₀H₃₄AuO₉PS, C₆H₁₁AuO₅S, or C₄H₅AuO₄S.

In another embodiment of the invention, the gold compound is a gold cluster complex, wherein multiple gold atoms and multiple peptides or proteins or polymers form a complex molecule. Preferably, the gold compound has a chemical formula Auₘ(SG)ₙ, wherein Au represents gold atoms, SG represents a small peptide, m is an integer between 3-200 and n is an integer between 2-220, more preferably, m is an integer between 10-43 and n is an integer between 10-42.

In another embodiment of the invention, SG represents a glutathione peptide (SEQ ID NO. 1) or SV peptide (SEQ ID NO. 6).

In another embodiment of the invention, the gold compound is selected from Au₁₀₋₁₂(SG)₁₀₋₁₂, Au₁₅(SG)₁₃, Au₁₈(SG)₁₄, Au₂₂(SG)₁₆, Au₂₂(SG)₁₇, Au₂₂(SG)₁₈, Au₂₅(SG)₁₈, Au₂₅(SG)₉, Au₂₉(SG)₂₀, Au₂₉(SG)₂₇, Au₃₀(SG)₂₈, Au₃₃(SG)₂₂, Au₃₅(SG)₂₂, Au₃₈(SG)₂₄, Au₃₉(SG)₂₄, Au₁₈(SG)₁₁, Au₂₁(SG)₁₂, Au₂₅(SG)₁₄, Au₂₈(SG)₁₆, Au₃₂(SG)₁₈, Au₃₉(SG)₂₃, and Au₄₃(SG)₃₇. Preferably, the gold compound is a gold cluster complex having a molecular formula Au₂₉(SG)₂₇, wherein SG represents a glutathione peptide (SEQ ID NO. 1) and Au represents a gold atom.

Yet in another embodiment of the invention, the coronavirus infection is COVID-19 and the coronavirus is SARS-CoV-2.

Another aspect of the invention relates to the use of a therapeutic composition in the manufacture of a medicament for treating or preventing a coronavirus infection in an animal.

In one embodiment of the invention, the use comprises the step of: preparing the therapeutic composition and administering sufficient amount of said therapeutic composition to said animal.

In another embodiment of the invention, said step of administering is through an intranasal spray, intraperitoneal injection, intramuscular injection, intravenous injection, or nasal inhaling method.

In another embodiment of the invention, said step of preparing a therapeutic agent further comprises the step of reacting gold (I) or gold (III) salt with glutathione peptide (SEQ ID NO. 1) solutions.

Yet in another embodiment of the invention, wherein the sufficient amount is in the range of 1 mg/kg.bw to 30 mg/kg.bw of the animal.

Further another aspect of the invention relates to a therapeutic composition for treatment of coronavirus infection in an animal, the coronavirus encoding a conserved papain-like main protease critical for its replication, comprising: a gold cluster compound stored in a liquid form having a molecular formula chemical Auₘ(SG)ₙ wherein Au represents gold atoms, SG represents a small peptide, m is an integer between 3-200 and n is an integer between 2-220 wherein SG represents glutathione peptide (SEQ ID NO. 1) or SV peptide (SEQ ID NO. 6) wherein said gold cluster compound functions as an active ingredient that both forms Au—S bond in the active pocket of the main protease and suppresses cytokine expressions in the animal’s body.

In one embodiment of the invention, the gold cluster compound is administered to said animal for at least 4 days.

In another embodiment of the invention, the gold cluster compound is administered to said animal through a nasal spray method.

In another embodiment of the invention, the gold cluster compound is administered to said animal through transdermal absorption.

For treatment of COVID-19, in one embodiment, a sufficient amount of liquid suspension of gold compound molecules is intranasally administered to COVID-19 patients to reduce the symptoms of COVID-19.

In another embodiment, a sufficient amount of gold compound molecules are administered to COVID-19 patients via parenteral injection or to reduce the symptoms of COVID-19.

### BRIEF DESCRIPTION OF THE DRAWINGS

The disclosed application will be described with reference to the accompanying drawings, which show important sample embodiments of the invention and which are incorporated in the specification hereof by reference, wherein:

FIG. 1A illustrates the gold cluster structure of a GA molecule.

FIG. 1B illustrates the structure of an AURANOFIN (AF) molecule.

FIG. 1C illustrates a coronavirus genome organization, replicase gene expression, and polyprotein processing.

FIG. 2A is the SDS-PAGE gel of purified M^{pro}-His tag protein and untagged SARS-CoV-2 M^{pro} in accordance with this application.

FIG. 2B is a size-exclusion chromatography of untagged M^{pro} protease in accordance with this application.

FIG. 3 is the mass spectrum of the purified M^{pro} protease in accordance with this application.

FIG. 4 is an X-ray crystal structure model of a gold compound-treated M^{pro} in accordance with this application.

FIG. 5 is a cartoon presentation of an X-ray crystal structure model of gold-S bonding in Domain I-III of an M^{pro} monomer. The enlarged views are of the Au(I)—S bond sites.

FIG. 6A shows the DFT models of Au binding pockets of Cys-145 and Cys-156 of M^{pro} in the presence of Au (I).

FIG. 6B shows the DFT models of geometrically relaxed structures of the binding pockets of M^{pro} encapsulating Au atoms, all Au—N atomic distances within 5 Å are labeled with corresponding distances, C, N, O, S, and Au atoms are displayed.

FIG. 7A shows the surface presentation comparison between the catalytic pocket Cys-145 of M^{pro} and the Au(I)—S bound state with the surrounding residues shown in sticks.

FIG. 7B shows the surface presentation comparison between the catalytic pocket Cys-156 of M^{pro} and the Au(I)—S bound state with the surrounding residues shown in sticks.

FIG. 8A shows the inhibition of M^{pro}protease activity and the suppression of virus replication activity (Panels A and C) by AF.

FIG. 8B shows the inhibition of M^{pro} protease activity and the suppression of virus replication activity (Panels B and D) by GA.

FIG. 8C shows the inhibition of M^{pro} protease activity by REMDESIVIR.

FIG. 9A shows the purified M^{pro} protease activity expressed in GA-treated HEK293F cells.

FIG. 9B shows the amount of gold contained in the purified M^{pro} protease from HEK293F cells treated with GA.

FIG. 10 shows the comparison of effects on cell viability on different cell lines by AF and GA respectively.

FIG. 11A shows the western blots of different cytokine expressions in macrophages RAW 264.7 cells with or without the presence of various concentrations of GA and FA gold compounds in accordance with this application.

FIG. 11B shows the western blots of different cytokine expressions in human bronchial epithelial cell line 16HBE cells with or without the presence of various concentrations of GA and FA gold compounds in accordance with this application.

FIG. 12A shows the quantitative analysis of different cytokine expressions in macrophages RAW 264.7 cells with or without the presence of various concentrations of GA and FA gold compounds in accordance with this application.

FIG. 12B shows the quantitative analysis of different cytokine expressions in human bronchial epithelial cell line 16HBE cells with or without the presence of various concentrations of GA and FA gold compounds in accordance with this application.

FIG. 13 shows the changes in body weight, RNA viral copies, and the pathology of the lungs of COVID-19 mice with or without the treatment of GA gold compound via intraperitoneal injection.

FIG. 14 shows the hematoxylin-eosin (HE) staining of the lungs from COVID-19 mice with or without the treatment of GA gold compound via intraperitoneal injection.

FIG. 15 shows the comparison of the fluorescence images stained for IL-6, IL-1β, TNF-α, and SARS-CoV-2 spike in the lungs of four COVID-19 mice in each group with or without the treatment of GA gold compound via intraperitoneal injection.

FIG. 16 shows the quantitative measurement of the various cytokines in the lungs of COVID-19 mice with or without the treatment of GA gold compound via intraperitoneal injection.

FIG. 17A shows the histopathological staining images of the lung tissue sections of normal BALB/c mice.

FIG. 17B shows the histopathological staining images of the lung tissue sections of COVID-19 BALB/c mice with NS (normal saline, 0.9% NaCl) treatment.

FIG. 17C shows the histopathological staining images of the lung tissue sections of COVID-19 BALB/c mice with GA gold compound treatment via intraperitoneal injection.

FIGS. 18A and 18B show histopathological stainings of various tissues of normal mice with or without GA treatment via intraperitoneal injection.

FIG. 19A shows the intranasal administering procedure of COVID-19 golden hamster rats.

FIG. 19B shows the hematoxylin-eosin (HE) staining of the lungs from COVID-19 rats with or without the intranasal administering treatment of GA gold compound.

FIG. 20A shows the comparison of the fluorescence images stained for IL-6, IL-1β, TNF-α, and SARS-CoV-2 spike in the lungs of four COVID-19 rats in each group with or without the intranasal administering treatment of gold cluster GA.

FIG. 20B shows the quantitative measurement of the various cytokines and SARS-CoV-2 spike levels in the lungs of COVID-19 rats with or without the intranasal administering treatment of GA gold compound.

FIG. 21A shows the molecule structure and formula of Au₂₅(SV)₉, SV here represents the small peptide CysCysTyrGlyGlyProLysLysLysArgLysProGly (SEQ ID NO. 6).

FIG. 21B shows the suppression of M^{pro} protease activity by Au₂₅(SV)₉.

FIG. 22 shows the suppression of M^{pro} protease activity by Au₁₀(SG)₁₀.

FIG. 23 shows the suppression of M^{pro} protease activity by Au₄₃(SG)₃₇.

### DETAILED DESCRIPTION OF SAMPLE EMBODIMENTS

The numerous innovative teachings of the present application will be described with particular reference to presently preferred embodiments (by way of example, and not of limitation). The present application describes several embodiments, and none of the statements below should be taken as limiting the claims generally.

For simplicity and clarity of illustration, the following figures illustrate the general manner of construction, description and details of well-known features and techniques that may be omitted to avoid unnecessarily obscuring the invention. Additionally, elements in the figures are not necessarily drawn to scale, some areas or elements may be expanded to help improve understanding of the embodiments of the invention.

In the present application, the animal may also be human being.

The terms “first,” “second,” “third,” “fourth,” and the like in the description and the claims, if any, may be used for distinguishing between similar elements and not necessarily for describing a particular sequential or chronological order. It is to be understood that the terms used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those elements, but may include other elements not expressly listed or inherent to such process, method, article, apparatus, or composition.

The term “COVID-19” refers to the infectious disease that is at least RT-qPCR tested positive for the presence of severe acute respiratory syndrome coronavirus 2 (SARS-CoV-2). COVID-19 symptoms may include high fever, cough, trouble breathing, persistent pain or pressure in the chest, new confusion, inability to wake or stay awake, bluish lips or face, and/or organ failure. Coronaviruses are a family of enveloped, positive-strand RNA viruses responsible for a wide range of diseases in a diverse range of animal hosts. Seven human coronaviruses (HCoVs) have been identified to cause respiratory diseases of varying severities: HCoV-OC43, HCoV-229E, HCoV-NL63, HCoVHKU1, SARS-CoV, MERS-CoV and SARS-CoV-2. Among these seven HCoVs, four (HCoV-OC43, HCoV-229E, HCoV-NL63 and HCoVHKU1) are common co-circulating, seasonal coronaviruses that cause mild respiratory tract infections generally associated with cases of the common cold. Unlike other coronaviruses, SARS-CoV-2 disease can specifically cause systemic inflammation which can develop further into acute cardiac injuries, sepsis, abnormal organ functions, and heart failure. Other distinctive clinical features of SARS-CoV-2 include sore throat, hypoxaemia, dyspnoea, sneezing, and diarrhoea.

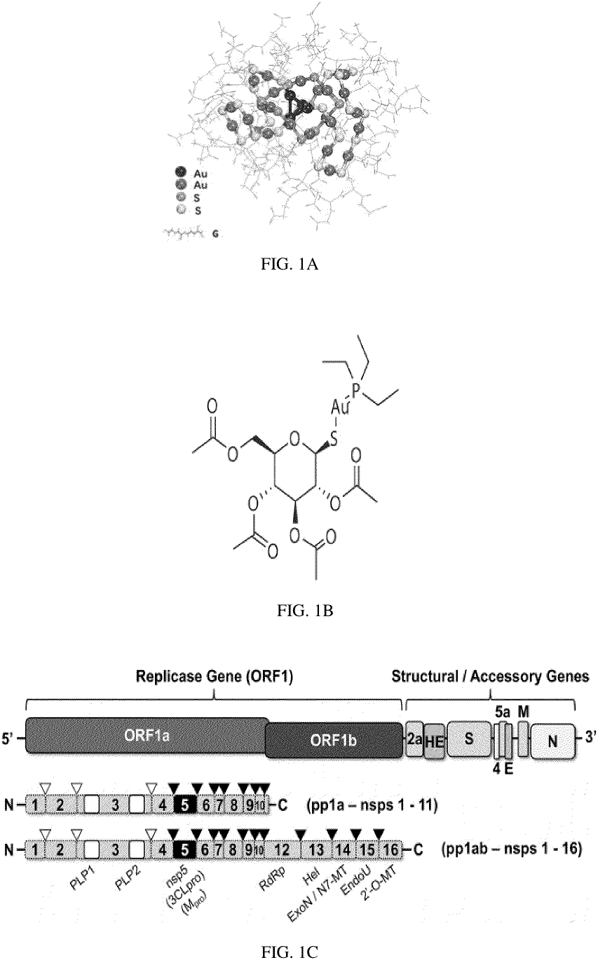

The term “gold cluster compound” refers to gold-atom cluster molecules comprised of a defined number of multiple gold atoms in a structured complex with a defined number of multiple gold binding molecules that have a thiol or selenol or phosphine or amine or arginine group, and such gold cluster molecules can characteristically emit fluorescent lights under excitation lights. The method of making such “gold cluster” molecules is described in US 8383919 B2 to XueyunGao, and the entirety of which is hereby incorporated by reference. These gold cluster compounds have been found to have a variety of biological effects. For example, gold-cluster molecules were found to cause the apoptosis of cancer cells, see e. g. US 9090660 B2 to XueyunGao, the entirety of which is hereby incorporated by reference; to mitigate bone loss in animals, see e. g. US 10029019 B1 to XueyunGao, the entirety of which is hereby incorporated by reference; to mitigate rheumatoid arthritis symptoms for rheumatoid arthritic animals, see e. g. US 9993562 B2 to XueyunGao. In this instant application, gold cluster compounds can be represented by the chemical formula of AuₓPeptide_{y}, wherein Au represents gold atom, peptide represents peptide or/and protein molecules, x=3-200, y=2-220. Preferably, the gold cluster compounds is Auₘ(SG)ₙ, wherein m=3-200 and n=2-220, wherein Au represents a gold atom, SG represents a small peptide, m is the number of gold atoms and n is the number of peptides in gold cluster complex. Preferably, m=10-43 and n=10-42.In an embodiment of present disclosure, there is providedAu₁₀₋₁₂(SG)₁₀₋₁₂, Au₁₅(SG)₁₃, Au₁₈(SG)₁₄, Au₂₂(SG)₁₆, Au₂₂(SG)₁₇, Au₂₂(SG)₁₈, Au₂₅(SG)₁₈, Au₂₅(SG)₉, Au₂₉(SG)₂₀, Au₂₉(SG)₂₇, Au₃₀(SG)₂₈, Au₃₃(SG)₂₂, Au₃₅(SG)₂₂, Au₃₈(SG)₂₄, Au₃₉(SG)₂₄, Au₁₈(SG)₁₁, Au₂₁(SG)₁₂, Au₂₅(SG)₁₄, Au₂₈(SG)₁₆, Au₃₂(SG)₁₈, Au₃₉(SG)₂₃ or Au₄₃(SG)₃₇. In this application, GA refers to the gold cluster molecule having a molecule formula Au₂₉(SG)₂₇ with a measured molecular weight of 13,983 Da by electrospray ionization mass spectrometry, and it comprises of 29 Au atoms and 27 glutathione molecules formed in a cluster structure, for illustration purpose, as illustrated in FIG. 1A. Whereas glutathion is a short peptide having a three amino acid composition of Glu-Cys-Gly (SEQ ID NO: 1). GA is an example gold cluster compound in this application, which is found to inhibit SARS-CoV-2 main protease activity both in vitro and in vivo. Another example gold cluster compound is Au₂₅(SV)₉, where SV represents the synthetic small peptide CysCysTyrGlyGlyProLysLysLysArgLysProGly (SEQ ID NO. 6).

The term AURANOFIN (AF) is a prescribed medicine drug refers to the gold salt having a molecule formula C₂₀H₃₄AuO₉PS⁰ with a structure illustrated in FIG. 1B. AF is an FDA approved drug for the treatment of rheumatoid arthritis and is classified by the World Health Organization as an anti-rheumatic agent. AF in this application is found to inhibit SARS-CoV-2 main protease activity both in vitro and in vivo. Another rheumatoid arthritis gold compound aurothioglucose was also found to be able to inhibit M^{pro} protease activities (data not shown).

The term “SARS-CoV-2” strain used in this research refers to a virus that is isolated from a COVID-19 patient (BetaCoV/Wuhan/IVDC/-HB -01/2020, EPI_ISL_402119) and passaged on Vero cells. In particular, the viral RNA was extracted from 100 µL supernatant of infected cells using the automated nucleic acid extraction system (TIANLONG, China) by following the manufacturer’s recommendations. For SARS-CoV-2 virus detection was performed using the One Step PrimeScript RT-PCR kit (TAKARA, Japan) on the LIGHTCYCLER 480 REAL-TIME PCR system (Roche, Rotkreuz, Switzerland). The replicase gene of SARS-CoV-2 was used for the detection which comprises two open reading frames, ORFla and ORF1ab. The primers targeting SARS-COV-2 ORFla were used in the study: Forward primer (SEQ ID NO. 2): 5′-AGAAGATTGGTTAGATGATGATAGT-3′; Reverse primer (SEQ ID NO. 3): 5′-TTCCATCTCTAATTGAGGTTGAACC-3′; Probe (SEQ ID NO. 4): 5′-FAM-TCCTCACTGCCGTCTTGTTGACCA-BHQ1-3′.

The term “M^{pro}” refers to SARS-CoV-2 main protease, which is the papain-like protease(s) responsible for processing the polyproteins that are translated from SARS-CoV-2 viral RNA. “M^{pro}” is also called coronavirus protease nsp5 or 3CL^{pro}that is an approximately 30 kDa, three-domain cysteine protease conserved in structure and function in all known coronaviruses and serves as the main protease for proteolytic processing of the replicase polyproteins (ppla and pplab). Typically, coronaviruses code for two or three proteases to process the replicase polyprotein: one or two papain-like proteases (PLPs) encoded within nsp3, and one main protease, nsp5 (3CL^{pro} or M^{pro}). PLPs are responsible for cleavage events between nsp1 and the N terminus of nsp4, whereas all remaining pp1a/pp1ab cleavage events are mediated by nsp5. The name ‘main protease’, or M^{pro}, refers to the critical role of this protease in coronavirus gene expression and replicase processing, and its other name 3C-like protease (3CL^{pro}) refers to the similarities between this protease and 3C proteases seen in picorna viruses, namely their similar substrate specificities and core structural homology. Among coronaviruses, nsp5 proteases within the same genus generally exhibit sequence identity of greater than 80% whereas proteases in different genera are far more divergent with sequence identity much closer to 50% despite high tertiary and quaternary structural conservation, especially in domains 1 and 2. Sequence analysis of the SARS-CoV and SARS-CoV-2 proteases reveals only 12 residue differences (approximately 96% identity) spread throughout the structure of the protease, with the majority of these residues distant from the active site (including along the distal surface of domain 1 and within domain 3), which strongly supports the prospect of developing active-site inhibitors that target both proteases.

The term “intranasal administering” refers to the process of dropping or dripping or spraying drug substance into nasal cavities where nasal mucosa absorbs the drug substance.

The surface of the nasal mucosa in humans is around 150 cm², a tissue that is well supplied by blood vessels. This ensures rapid absorption of most of the drug, allowing generating high systemic blood levels and avoiding the first-pass metabolism which needs to be taken into account following oral administration. In this application, the efficiency of intranasal administration is tested.

FIG. 1C shows the coronavirus open reading frames (ORFs) associated with replication (replicase gene; ORF1a/ORF1b) and the structural and accessory genes. The two variant polyproteins (ppla and pplab) translated from the replicase gene are shown with the non-structural protein domains of the polyprotein-labelled and the proteolytic cleavage sites marked with arrows. Three proteases mediate the proteolytic processing of the replicase polyproteins (PLP1, PLP2, and nsp5 (3CL^{pro}/M^{pro})), and white arrows for PLPs and black arrows for nsp5 for each cleavage site correspond to the protease responsible for mediating its cleavage, wherein PLP stands for papain-like protease; RdRp stands for RNA-dependent RNA polymerase; Hel stands for helicase; ExoN stands for exonuclease; N7-MT stands for N7-methyltransferase; EndoU stands for endoribonuclease; 2′-O-MT stands for 2′-O-methyltransferase.

Typically, immediately upon entry into a host cell, the virus translates its replicase gene (ORF1) which consists of two large, overlapping ORFs, ORFla and ORF1ab. Located at the end of ORFla is a ribosome frame-shifting sequence consisting of an RNA pseudoknot that causes the co-translation of two large polyprotein precursors of differing lengths, ppla and pplab. Polyprotein ppla contains non-structural proteins (nsps) 1-11, and polyprotein pplab comprises the complete translated coding region of nsps 1-16. Essential for virus replication is the proteolytic processing of these polyproteins by virus-encoded proteases to yield the mature and functionally active replication machinery of the virus. Once proteolytically processed, the translation products of ppla collectively modulate host cell factors and help prepare the cell for viral RNA synthesis through the formation of replication complexes, while the C-terminal translation products of pplab largely catalyze and/or regulate the processes of RNA replication and transcription driven by the viral RdRp (nsp12). Replication complexes assemble on virus-induced membrane structures such as double-membrane vesicles and convoluted membranes driven by transmembrane nsps 3, 4, and 6. The active replication complex promotes the continuous and discontinuous synthesis of negative-sense RNA templates, which are subsequently used to drive the formation of genomic copies and a nested set of subgenomic RNAs from the downstream ORFs encoding structural and accessory proteins, respectively. Following replication of genomic and subgenomic RNA on double-membraned vesicles, structural proteins like the spike (S), envelope (E), matrix (M), and nucleocapsid (N) proteins are translated by existing positive-strand subgenomic RNAs. S, E, and M become glycosylated within the Golgi before localizing to the endoplasmic reticulum-Golgi intermediate compartment (ERGIC) to be assembled into virions.

Proteolytic processing acts as a key regulatory mechanism in the expression of the coronavirus replicase proteins, blocking this process has been demonstrated to block viral replication entirely. Background information can be found in Roe et al., “Targeting novel structural and functional features of coronavirus protease nsp5 (3CLpro, Mpro) in the age of COVID-19,” Journal of General Virology, PMID, 33507143, DOI 10.1099/jgv.0.001558 (Jan. 28, 2021), the entirety of which is therefore incorporated by reference.

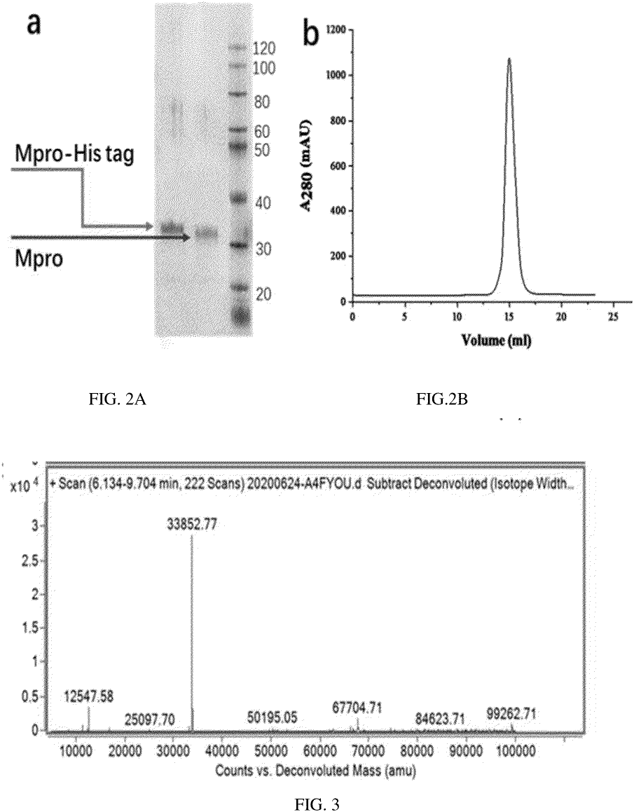

In the present application purified SARS-CoV-2 M^{pro} protein was obtained through cloning and expression of M^{pro} gene in E. coil. The full-length gene encoding SARS-CoV-2 M^{pro} was optimized and synthesized for Escherichia coli (E. coil) expression through GENEWIZ system. The gene was cloned into a modified pET-28a expression vector with an N-terminal (His) 6-tag followed by a Tobacco etch virus (TEV) cleavage site. The construct was confirmed by DNA sequencing. The plasmid was further isolated and transformed into the Escherichia coli Rosetta (DE3) expression strain of Invitrogen Inc. The E. coli cells containing the plasmids above were grown to an OD600 of 0.8 and induced with isopropyl β-D-thiogalactopyranoside (IPTG) to a final concentration of 0.5 mM at 16° C. for 14 hrs. E.coli cells were then harvested by centrifugation at 4600 g, re-suspended in lysis buffer (120 mM Tris/HCl, pH 8.0, 20 mM imidazole and 300 mM NaCl), and lysed by French press, the lysate was centrifuged at 15 000 g for 50 min. Then the supernatant was loaded onto a Ni-NTA column pre-equilibrated with lysis buffer and washed with 20 mM Tris/HCl, pH 8.0, 300 mM NaCl, and 50 mM imidazole. The protein was eluted in 20 mM Tris/HCl, pH 8.0, 150 mM NaCl and 300 mM imidazole. TEV protease was added to the His tag fused protein solution and dialyzed overnight into anion-exchange chromatography buffer A (20 mM Tris/HCl, pH 8.0, 20 mM NaCl, 1 mM DTT, 1 mM EDTA) to cleave off the His(6) tag. The tag-cleaved protein was further purified using a Resource-Q column of AKTA fast protein liquid chromatography of GE Healthcare, Inc by elution with a linear gradient of 20-500 mM NaCl, 20 mM Tris/HCl, 1 mM EDTA, 1 mM DTT, and pH 8.0. The purity of the obtained Mpro was analyzed by SDS/PAGE at each step. The purified and concentrated SARS-CoV-2 M^{pro} was stored in 20 mM Tris-HCl (pH 7.3), 20 mM NaCl, 1 mM DTT, and 1 mM EDTA for enzyme activity assays and crystallization. FIG. 2A shows the SDS/PAGE gel electrophoresis of the purified M^{pro} protein and FIG. 2B shows the size-exclusion chromatography profile of M^{pro} after the cleavage of the His(6) tag.

Five µl of the purified M^{pro} protein was used for Liquid chromatography-mass spectrometry analyses in positive-ion mode with a quadrupole-time-of-flight mass spectrometer combined with a high-performance liquid chromatograph for detecting the molecular weight of the purified M^{pro}protein. Mass deconvolution was performed using AGILENT MASSHUNTER Qualitative Analysis B.06.00 software with BIOCONFIRM WORKFLOW. The purified M^{pro} protein mass spectroscopy profile is shown in FIG. 3.

The purified M^{pro} protein was used for crystallization at 22° C. using the sitting-drop vapor-diffusion technique. About 0.7 µl 6 mg/ml protein solution mixed with an equal volume of reservoir solution was used for growing crystals. Initial crystals were found under the crystallization conditions of the PEG/Ion Screen Kit of CRYSTAL SCREEN by Hampton Research. After optimization, the best crystals of M^{pro} protein were obtained under the condition of 200 mM KF and 15% PEG 3350 after 4 to 5 days. For gold compound treatment, crystals of M^{pro} proteins were further soaked in reservoir solutions in the presence of 10 mM GA gold cluster compound solution or 10 mM AURANOFIN compound solution for over 15 hr. M^{pro} crystals treated with GA or AF were then X-ray analyzed for structure changes.

Prior to data collection, all crystals were cryo-protected by plunging them into a drop of reservoir solution supplemented with 10-20% glycerol, then flash frozen in liquid nitrogen. The X-ray diffraction data were collected at the beamlines in Shanghai Synchrotron Radiation Facility and were processed using software HKL3000 or XDS. The initial phase was determined by molecular replacement method using the program PHASER from CCP4 program suit, with the crystal structure of SARS-CoV-2 main protease M^{pro} in complex with an inhibitor N3 (PDB entry 6LU7) as the initial model. The structure refinement was carried out using PHENIX and REFMAC, model building was carried out by COOT, and MOLPROBITY was used to validate the structure. The locations of Au (I) ions were identified according to the anomalous difference Fourier maps. Data collection and refinement statistics are listed in Table I. M^{pro} crystal structures were constructed using PYMOL as available at pymol.org.

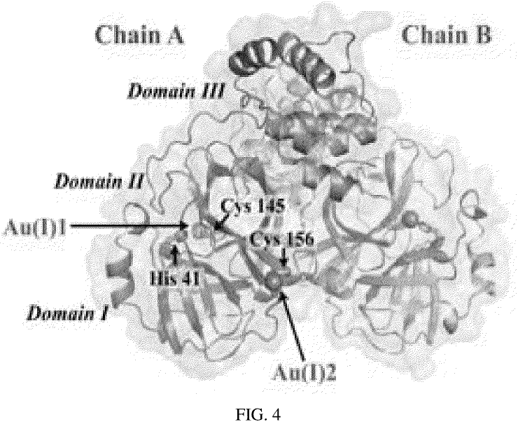

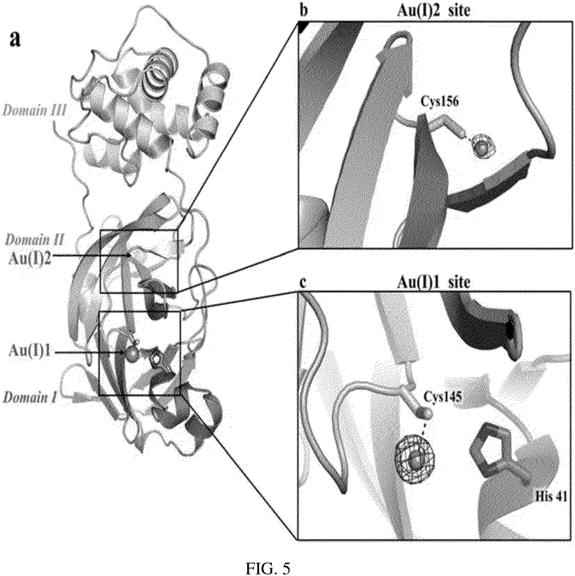

In reference to FIG. 4, the resulting SARS-CoV-2 M^{pro} crystal structures in the presence of either gold compounds (GA or AF) were very similar and shared most of the features as those of the crystal structures of the native M^{pro} previously determined. However, the densities of two Au(I) ions were found near the thiol residues of Cys145 and Cys156 of M^{pro} after the M^{pro} crystals were treated with either GA or AF compounds. The positions of these two Au(I) ions were confirmed by applying the anomalous difference Fourier maps, as shown in FIG. 5.

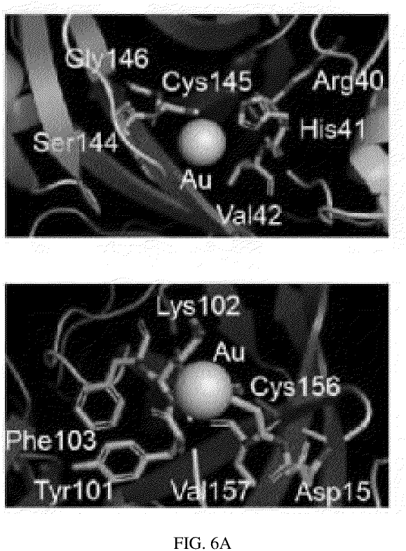

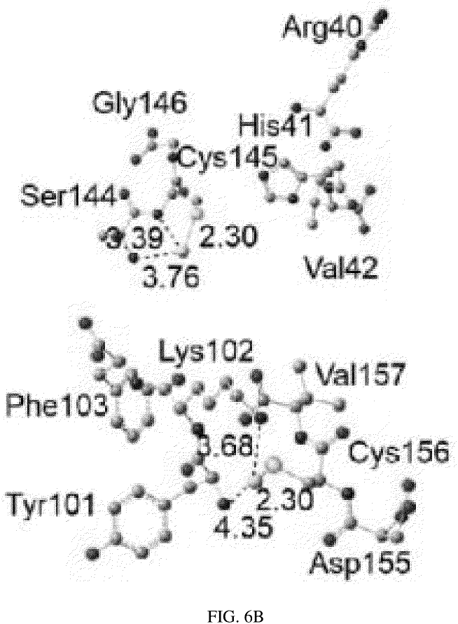

The binding energies between Au(I) and M^{pro} protein were calculated by density functional theory (DFT) calculations. According to the crystal structure shown in FIG. 4, M^{pro} protein has two binding pockets for Au(I) ion and each encapsulates one Au atom. The two Au atoms are binding with the S atoms of Cys145 and Cys156 respectively. To simulate the chemical environment of the Au atoms bound in the binding pockets, the residues within 5 Å from the Au atoms were considered. Specifically, Ser144, Cys145, Gly146, Arg40, His41, and Val42 in the first pocket, and Tyr101, Lys102, Phe103, Asp155, Cys15, 6 and Val157 in the second pocket were used for calculation. There are four peptide bonds in each pocket. To maintain the skeleton structures of the two pockets, positions of the C and N atoms of the peptide bonds were fixed and all the other atomic positions were allowed to be relaxed during the geometry optimizations. FIG. 6A and FIG. 6B. The B3LYP functional in conjunction with the SDD basis set for Au and the 6-31G (d, p) for nonmetal atoms were applied. The SDD pseudopotential was also applied for Au. During geometry optimizations, the SMD solvation model was utilized to model the water environment. All the calculations were carried out using GAUSSIAN 09 package. The bond dissociation energy (EBD) between Au and the protein binding pockets was calculated using the following equation,

### EBD = E_{Au} + E_{ligands} - E_{complex} (1)

Where E_{Au}, E_{ligands}, and E_{complex} were the total energies of the Au atom, ligands of the pocket, and the complex, respectively. E_{ligands} was obtained by single-point energy calculation based on the optimized geometries of complexes with the Au atom removed. DFT calculations confirmed that Au atoms preferred to form S—Au bond with the thiol groups of Cys145 and Cys156 of the M^{pro}protein binding pockets. In addition, the N atoms of Ser144 and Cys145 and those of Tyr101 and Lys102 have a distance within 5 Å from the corresponding Au atoms, suggesting there may be considerable electrostatic interactions between the respective N atoms and Au atoms. The bond dissociation energies (EBD’s) between Au and the two pockets are calculated to be respectively 46.1 kcal mol⁻¹ and 26.5 kcal mol⁻¹. Such large EBDs suggest that the respective Au atoms are firmly locked inside the pockets, which can cause the effective inhibition of the proteinase activity of M^{pro}.

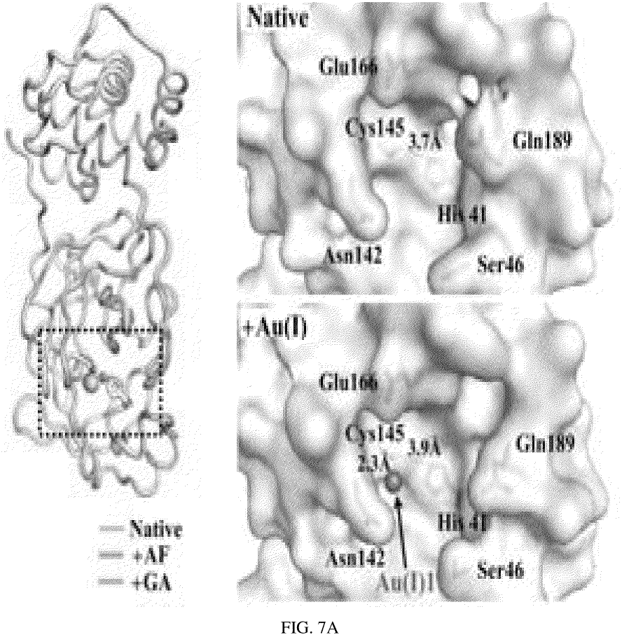

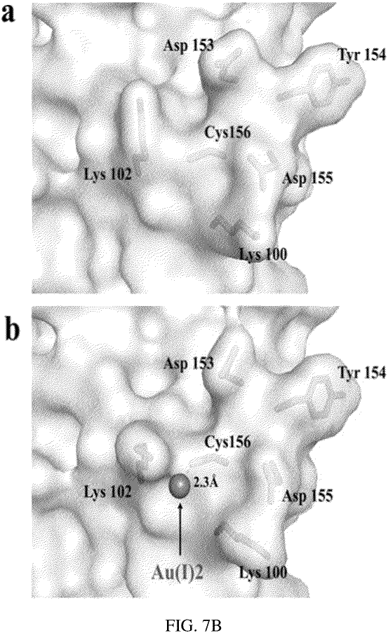

In references to FIGS. 7A and 7B, the Au(I)-Cys145 and Au(I)-Cys156 interactions are illustrated by superpositions of the crystal structures of AF-treated M^{pro}, GA-treated M^{pro}and the untreated M^{pro}. The surface presentations and the surrounding residues of the catalytic sites of native and Au(I)—S bound M^{pro} are shown to form binding pockets.

TABLE I

Crystal X-ray Data collection and refinement statistics

| | M^{pro}-AF treated | M^{pro}-GA treated | M^{pro}-Native |
|---|---|---|---|
| Data collection | | | |
| Wavelength (Å) | 0.86 | 0.98 | 0.98 |
| Space group | C2 | C2 | C2 |
| Cell dimensions | | | |
| a, b, c (Å) | 114.3, 54.0, 44.7 | 113.8, 53.8, 44.6 | 113.9, 53.8, 44.7 |
| a, β, γ (°) | 90.0, 101.8, 90.0 | 90.0, 102.0, 90.0 | 90.0, 101.5, 90.0 |
| Resolution (Å) | 50-2.75 (2.90- | 50-1.72 (1.75- | 50-1.77 (1.80-1.77)^{a} |
| | 2.75)^{a} | 1.72)^{a} | |
| R_{merge} | 0.094 (0.154) | 0.089 (0.823) | 0.070 (0.542) |
| < I/σ(I)> | 14.0 (7.0) | 33.8 (2.2) | 24.3 (2.4) |
| Completeness (%) | 96.4 (80.9) | 99.8 (100.0) | 99.3 (95.9) |
| Redundancy | 5.4 (3.4) | 6.2 (6.0) | 6.6 (4.9) |
| Resolution (Å) | 50-2.75 | 50-1.72 | 50-1.77 |
| No. reflections | 6,781 | 27,877 | 25,365 |
| R_{work}/R_{free} | 0.194/0.228 | 0.199/0.237 | 0.206/0.248 |
| Protein | 2,329 | 2,329 | 2,329 |
| Au | 2 | 2 | 0 |
| Water | 110 | 234 | 260 |
| B-factors | 34.8 | 37.8 | 24.7 |
| Rmsd bond length (Å) | 0.007 | 0.009 | 0.008 |
| Rmsd bond angle (°) | 1.0 | 1.0 | 1.0 |
| Favored (%) | 97.7 | 97.7 | 99.0 |
| Allowed (%) | 2.3 | 2.3 | 0.7 |
| Outliers (%) | 0 | 0 | 0.3 |

^{a}The values in parenthesis mean those of the highest resolution shell.

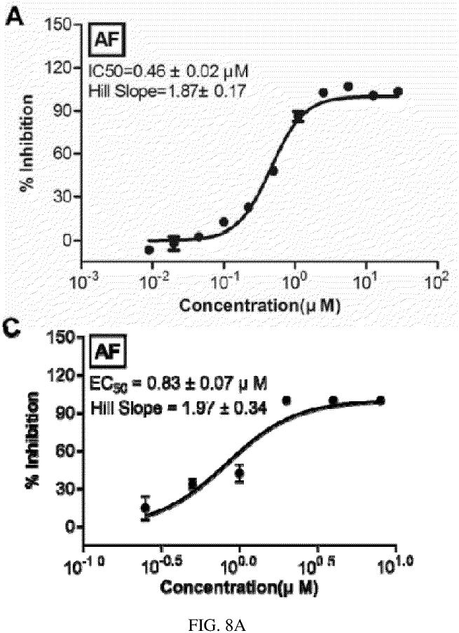

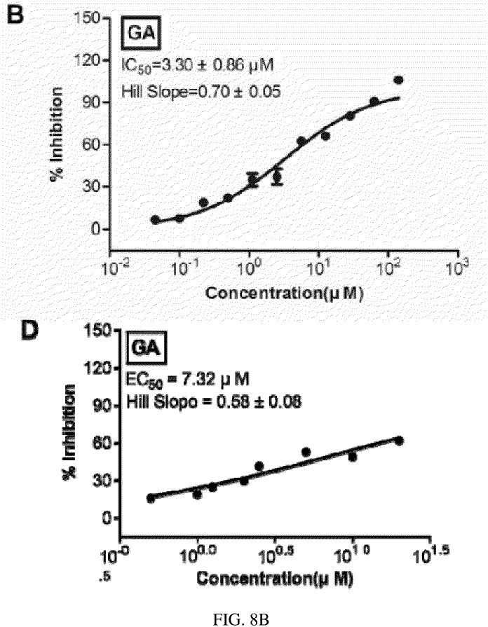

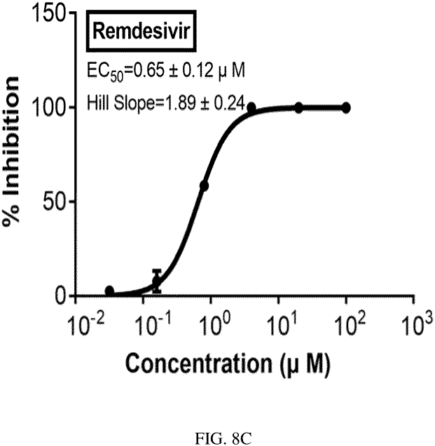

Referring to FIG. 8A and FIG. 8B, the inhibitory effects of GA and AF on protease activities of M^{pro} were measured according to the method by V. Grum-Tokars, et al., “Evaluating the 3C-like protease activity of SARS-Coronavirus: Recommendations for standardized assays for drug discovery,” Virus Research133, 63-73 (2008), the entirety of which is therefore incorporated by reference. Eleven Different concentrations of GA or AF were added into M^{pro} protease reaction mixture that contained 0.5 µMM^{pro} protein, 20 µM substrate (SEQ ID No: 5) (EDNAS-Glu)-Ser-Ala-Thr-Leu-Gln-Ser-Gly-Leu-Ala-(Lys-DABCYL)-Ser. M^{pro} activity was measured by fluorescence resonance energy transfer (FRET) assay. Fluorescence intensity was monitored by the multimode plate reader from Bio-Rad with excitation at 340 nm and emission at 535 nm. All experiments were performed in triplicates. The fluorescence-labeled substrate, (SEQ ID No: 5) (EDNAS-Glu)-Ser-Ala-Thr-Leu-Gln-Ser-Gly-Leu-Ala-(Lys-DABCYL)-Ser, was derived from the auto-cleavage sequence of the viral protease and was chemically modified for enzyme activity assay.

EC50 measurements with SARS-CoV-2 were performed under biosafety level 3 (BSL-3) conditions at the Chinese Center for Disease Control and Prevention, China. Vero cells were infected with SARS-CoV-2 at a multiplicity of infection (MOI) of 0.015 diluted in DMEM/F12 without FCS at 37° C. for 1 h. Cells were washed with DMEM/F12 with 10% FCS and supplemented with AURANOFIN or gold cluster GA in different concentrations. For solvent control, cells were only treated with 1% DMSO 48 hours after infection (h.p.i.), cells supernatant were collected and virus RNA samples were subjected to qRT-PCR measurement. All experiments were performed in triplicate.

In FIG. 8A, panel A shows the IC50 curve of AF, and panel B shows the EC50 of AF. The inhibition of 50% purified M^{pro} protease activity (IC50) reached about 0.46 µM AF. EC50 was tested on SARS-CoV-2 replication in Vero cells in the Biosafety Level-3 Lab of China CDC, and panel B in FIG. 8A shows that the EC50 of AF was about 0.83 µM. The testing method was according to A. Pizzorno, et al., “Characterization and treatment of SARS-CoV-2 in nasal and bronchial human airway epithelia,” Cell Rep. Med. 1, 100059 (2020), the entirety of which is incorporated by references.

Compared to the reported IC 50 for the known COVID-19 drug EBSLEN is 0.67 µM, AURANOFIN is shown to be a strong inhibitor for M^{pro} protease activity. Whereas the EC50 for the known COVID-19 drug REMDESIVIR is about 0.65 µM (FIG. 8C), EC50 of AURANOFIN (about 0.83 µM) is close to the effectiveness of REMDESIVIR.

Similar measurements were performed using gold cluster compound GA, as shown in FIG. 8B, panel A shows that the inhibition of 50% purified M^{pro} protease activity (IC50) reached about 3.30 µM of GA, and panel B shows that the inhibition of 50% SARS-CoV-2 replication in Vero cells reached about 7.32 µM of GA.

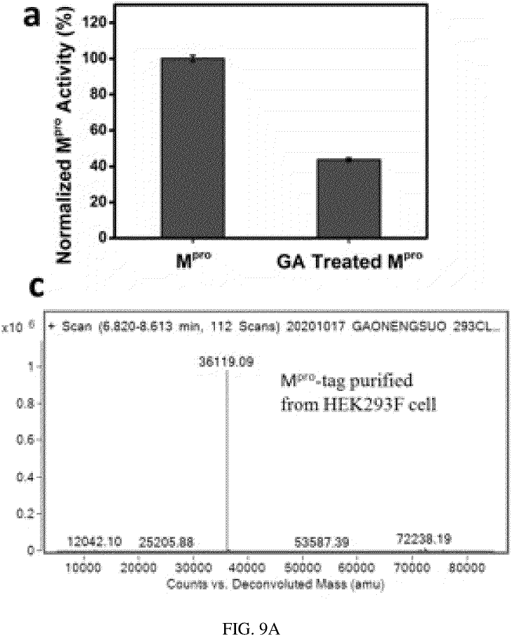

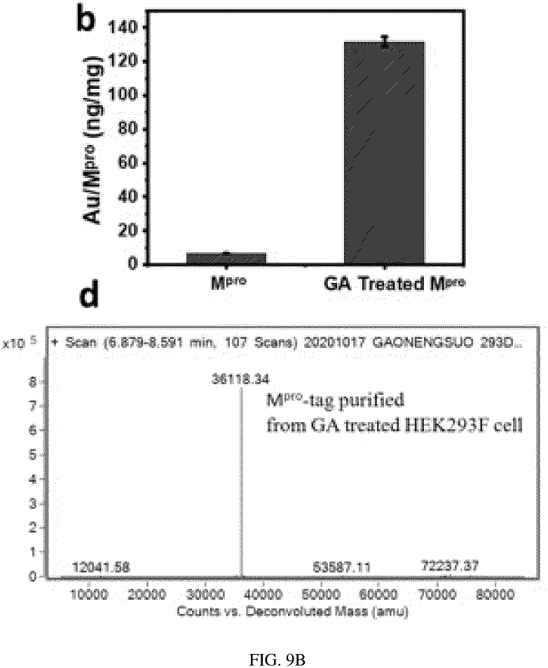

The inhibitory effect of GA on M^{pro} protease activity in vivo was further tested on HEK293F cells transiently transfected with a plasmid containing strep-tagged SARS-CoV-2 M^{pro} gene. The M^{pro} gene was expressed for 24 hrs in HEK293F cells before GA was added to the culture medium for a final concentration of 500 µM, and cells were cultured for an additional 24 hrs. After cells were harvested, SARS-CoV-2 M^{pro} proteins were extracted from GA-treated HEK293F cells and were purified and analyzed for enzyme activity by MASS spectroscopy. As shown in FIG. 9A, in panel A, the M^{pro} protease extracted from GA-treated HEK293F cells was about 40% as that of control M^{pro} activity. FIG. 9B confirms that the M^{pro} protein purified from GA-treated HEK293F cells contained gold atoms. There was about 120 ng Au per mg M^{pro} protein extract. However, the mass spectroscopy of M^{pro}-tag purified from untreated HEK293F cells showed a matching molecular weight of about 36119 Da, while the mass spectroscopy of M^{pro}-tag purified from GA-treated HEK293F cells showed a band of molecular weight of about 36118 Da, the missing Au signal in the mass spectroscopy of M^{pro}-tag purified from GA treated HEK293F cells was probably due to the laser ablation that would have broken the Au—S bond of the samples.

Recently Rothanet al reported that AF well inhibited SARS-CoV-2 replication in infected Huh cells and the EC50 of AF was about 1.4 µM, and they speculated that inhibition of SARS-CoV-2 replication might be induced by gold compound suppressing the thioredoxin reductase activity and inducing ER stress of host cells. See H. A. Rothan, et al, “The FDA-approved gold drug Auranofin inhibits novel coronavirus (SARS-COV-2) replication and attenuates inflammation in human cells,” Virology 547, 7-11 (2020). However, based on the crystal structure studies in this application and the M^{pro} activity data reported herein, it is more likely that the gold compounds inhibit SARS-CoV-2 replication via Au(I) binding to Cys145 and Cys 156 of M^{pro}, causing the inhibition of its activity in host cells.

To test whether GA and AF are toxic to normal cells, Vero E6, RAW264.7, and 16HBE cell lines were tested. Various doses of AURANOFIN or GA gold cluster were added into cell culture media respectively. After 48 hours of incubation, cell viability was checked by CCK8 (Beyotime, China) following the manufacturer’s instruction, all studies were carried out in triplicate. All cell lines were obtained from ATCC with authentication service.

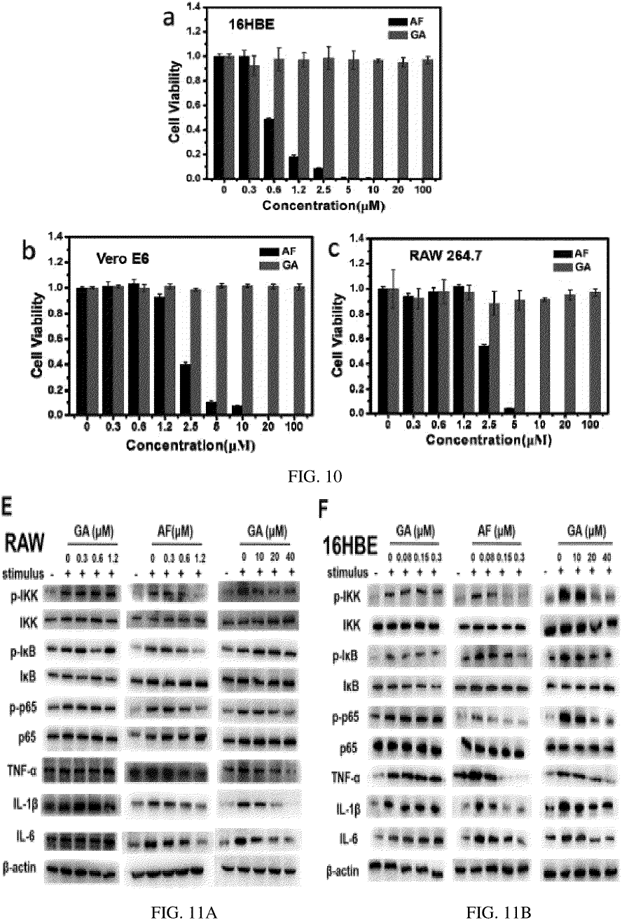

Referring to FIG. 10, for human bronchial epithelial cells (16HBE), the CC50 of AF was about 0.6 µM while gold cluster GA showed no cell toxicity even at a concentration of 100 µM in cell cultures for 48 hrs. For Vero E6 cell, the CC50 of AF was about 2.2 µM while gold cluster GA showed no cell toxicity even at 100 µM in cell cultures for 48 hrs. For macrophage RAW264.7 cells, the CC50 of AF was about 2.4 µM and gold cluster GA again showed no cell toxicity even when its dose was increased to 100 µM in cell cultures for 48 hrs. For in vivo toxicity, in terms of mice acute toxicity, the intraperitoneal LD50 for AF was known to be about 33.8 mg/kg. In terms of rat acute toxicity, the intraperitoneal LD50 for AF was known to be about 25.5 mg/kg and it was about 288 mg/kg.bw for GA.

The in vivo toxicity LD50 was measured in mice as follows. 100 adult female BALB/c mice for experiments were conducted in compliance with regulations of the National Act on the use of experimental animals (China) and were approved by the Institutional animal care and ethic committee at the Chinese Academy of Sciences (approved No. SYXK (jing) 2014-0023). Their weights ranged between (18 g-22 g). Mice were housed in plastic cages, each cage contained 10 mice. Animals were kept at a controlled temperature of 25 ± 2° C. for 12 hours under light and 12 hours dark cycle throughout the experiment. The LD50 was studied by a “staircase method” with increasing doses of FA or GA. Ten doses of 100, 200, 300, 400, 500, 600, 700, 800, 900, and 1000 mg/kg b.wt per body weight were given to 10 groups of mice (10 in each) for the determination of intraperitoneal LD50 in female mice. Animals were observed for the 2, 6, and 24 hours for any toxic symptoms. After 24 hours, dead animals were counted in each group and LD50 was determined by the method of Karber. In this study, no mice were dead within 24 hours after various amounts of GA were injected.

In the rheumatoid arthritis rat/mice treatment model, when oral AF was given at a dose of 6-9 mg/kg.bw and an intraperitoneal injection of GA was given at 5 mg/kg.bw, both of the two treatments doses of AF and GA showed significant suppression of inflammatory cytokine levels and were observed to achieve a similar outcome for rheumatoid arthritis treatment. However, the toxicity data on the cellular level, on mice and rats in vivo, and on rheumatoid arthritis model mice/rat treatments, all suggest that gold cluster GA is a safer form of the gold compound, and it may be a better choice as a drug and of higher safety than AF when electing them as treatments COVID19 patients.

TABLE II

Mice/Rat Acute Toxicity and Cytotoxicity of GA and AF

| Animal/cell | Gold compounds | |
|---|---|---|
| | GA | AF |
| BALB/c Mice | LD50 >1000 mg/kg.bw | LD50, about 33.8 mg/kg.bw |
| SD Rat | LD50,about 288 mg/kg.bw | LD50, about 25 mg/kg.bw |
| epithelial cell | CC50 31.9 µM | CC50, about 0.63 µM |
| Vero cell | CC50 33.84 µM | CC50 about 2.27 µM |
| Macrophage cell | CC50 43.2 µM | CC50 about 2.63 µM |

Most recently, a clinical study revealed that severe COVID-19 patients have a hyperinflammatory immune response associated with macrophage activation. See Y. Cao, et al. “Ruxolitinib in treatment of severe coronavirus disease 2019 (COVID-19): A multicenter, single-blind, randomized controlled trial,” J. Allergy Clin. Immunol. 146, 137-146 (2020). By using RA treatment drugs, RUXOLITINIB, to inhibit the activation of the NFκB pathway in macrophages, down regulation of the expression level of IL-6, IL-1β, and TNF-α were observed and the oxygenation and clinical status of most severe patients on supplemental oxygen were improved relatively rapidly.

Referring to FIG. 11A and FIG. 11B, the effects of gold compound AF and GA on cytokine expressions were measured using mircophage cell lines RAW 264.7 (FIG. 11A) and human bronchial epithelial cell 16HBE cell (FIG. 11B). RAW 264.7 or 16HBE cells were seeded into 6 well plates at a density of 2 × 10⁶cells/well. After incubation with or without the presence of TNFα (50 ng mL⁻¹) under different concentrations of AURANOFIN or gold cluster GA for 24 hrs, the cells were collected and lysed with RIPA buffer (50 mmol L⁻¹Tris-HCl, pH 7.4, 150 mmol L⁻¹NaCl, 1% Triton X-100, 1% sodium deoxycholate, 0.1% SDS, 1 mmol L⁻¹sodium orthovanadate, 50 mmol L⁻¹NaF, and 1 mmol L⁻¹ethylenediaminetetraacetic acid) along with protease inhibitor purchased from Roche Molecular Biochemicals. The collected cell lysates were centrifuged at 13000 rpm for 10 min, and the supernatants were stored for the subsequent Western Blot analysis. The protein concentrations of the supernatants were determined using a microplate spectrophotometer (SPECTRAMAX M4 of Molecular Devices, USA) at a wavelength of 595 nm. An equal quantity of proteins (50 µg) was separated by running on 10% SDS-PAGE and the separated proteins were transferred to a polyvinylidene fluoride (PVDF) membrane (0.45 µm, Millipore, USA). After blocking, the membranes were incubated with specific antibodies for COX-2 (from Cell Signaling Technologies, 12282, 1:1000), IL-1β (from Cell Signaling Technologies, 12703, 1:1000), IL-6 (from Cell Signaling Technologies, 12912, 1:1000), TNF-α (from Cell Signaling Technologies, 11948, 1:1000), phosphor-p65 (from Cell Signaling Technologies, 3033, 1:1000), p65 (from Cell Signaling Technologies, 3034, 1:1000), phosphor-IκBα (from Cell Signaling Technologies, 2859, 1:1000), IκBα (from Cell Signaling Technologies, 4812, 1:1000), IKKα (from Cell Signaling Technologies, 2682, 1:1000), IKKβ (from Cell Signaling Technologies, 8943, 1:1000), phosphor-IKKα/β (from Cell Signaling Technologies, 2697, 1:1000), this step was followed by incubation with an appropriate secondary antibody conjugated to horseradish peroxidase (Beyotime Biotechnology, China) to produce visible fluorescence for measurement.

As shown in FIG. 11A and FIG. 11B, relatively low dose of AF (1.2 µM) significantly suppressed IL-6, IL-1β, and TNF-α expression levels in both macrophage cells and human bronchial epithelial cells. Relatively high doses of GA (40 µM) also significantly suppressed the levels of IL-6, IL-1β, and TNF-α in both inflammatory macrophage cells and human bronchial epithelial cells.

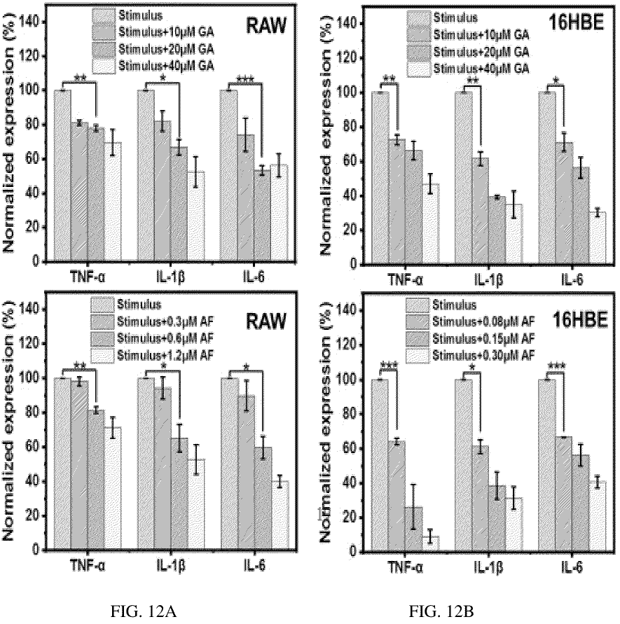

The dose-dependent cytokine expression suppressions are quantitatively shown in FIG. 12.

In inflammatory macrophages, the nuclear factor NFκB is the key signaling pathway that regulates the inflammatory mediator genes which involve the inflammatory factors to induce the activation of the IκB kinase (IKK) complex, causing subsequent degradation of IκB proteins, and releasing p-p65 which enters the nuclei to induce the expression of TNF-α, IL-1β, and IL-6. As shown by the Western blots, AF or GA treatment could decrease IKK phosphorylation level, causing suppressed IκB phosphorylation and the inhibition of p65 phosphorylation. In this case, AF at a low dose (1.2 µM) could inhibit phosphorylation of IKK, IκB, and p65, thus suppress the NFκB activation. GA at a relatively high dose (40 µM) could inhibit the phosphorylation of IKK, IκB, and p65, thus suppressing NFκB activation in inflammatory macrophage cells and human bronchial epithelial cells.

As reported in COVID-19 patients, virus-infected bronchial epithelial cells would activate NFκB pathway to express inflammatory cytokine, these cytokines would activate macrophages into inflammatory status. Shown in FIG. 11B, gold compounds were able to inactivate NFκB pathway and suppress the inflammatory cytokine expression level in inflammatory human bronchial epithelial cells. AF in low dose of (0.15 µM) and GA of high dose (20 µM) may have significantly inhibited the phosphorylation level of IKK, IκB, p65 to suppress NFκB activation, thus inhibiting IL-6, IL-1β, and TNF-αinflammatory cytokine expression in these bronchial epithelial cells.

### GA Treatment of COVID-19 Mice

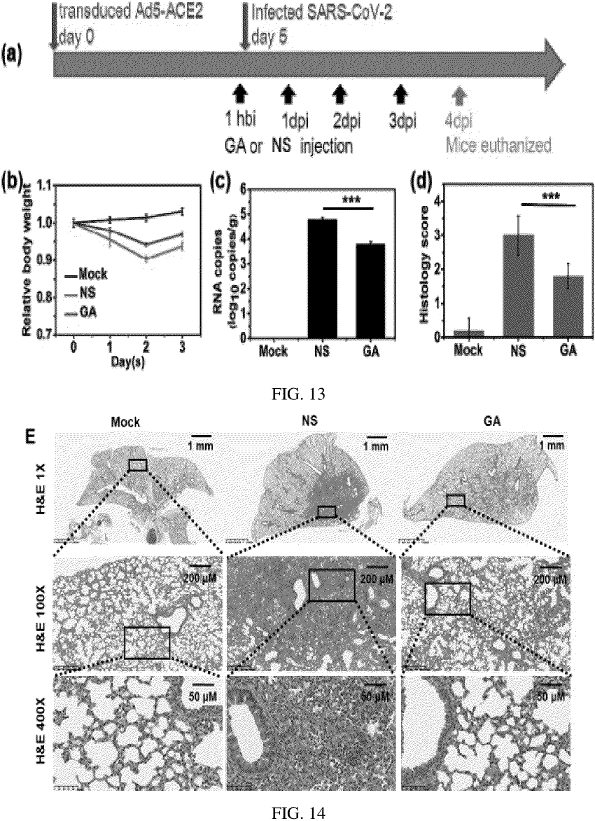

The COVID-19 mice model was generated following a recently reported method and detailed procedures are illustrated in FIG. 13. See J. Sun, et al, “Generation of a broadly useful model for COVID-19 pathogenesis, vaccination, and treatment,” Cell 182, 734-743 (2020).

In particular, pathogen-free 6eek-old female BALB/c mice were purchased from SiPeiFu Laboratory Animal Co (Beijing, China). All protocols were approved by the Institutional Animal Care and Use Committees of the National, Institute for Viral Disease Control & Prevention, and the Chinese Center for Disease Control and Prevention. The SARS-CoV-2 strains used in this research were isolated from COVID-19 patients (BetaCoV/Wuhan/IVDC/-HB -01/2020, EPI_ISL_402119) and passaged on Vero cells. The human serotype 5 adenoviral vector expressing human ACE2 under the control of the CMV promoter was a gift kindly provided by Dr. Zhao Juncun.

COVID-19 mice were generated as previously reported. See S. Du, et al., “Structurally resolved SARS-CoV-2 antibody shows high efficacy in severely infected hamsters and provides a potent cocktail pairing strategy,” Cell 183, 1-11 (2020). 12 mice were divided into 3 groups with four mice each, at day 0, mice were anesthetized with pentasorbital sodium and transduced intranasally with 2.5×10⁸ FFU of Ad5-ACE2 in 50 µL DMEM. Five days after transduction, 1 hr before infection, these mice received either a dose of 15 mg/kg GA intraperitoneal injection (i.p.) in a volume of 150 µL or an equivalent volume of Normal Saline (NS, 0.9% sodium chloride) administered to control mice. These mice were then infected intranasally with SARS-CoV-2 (1×10⁵ PFU) in a total volume of 50 µL DMEM. Infected mice continued to receive either GA or Normal Saline (NS) i.p. treatment for three days. All mice were weighed every day and euthanized at 4dpi. The mice lungs were collected and weighed, and lung homogenates were prepared in NS (0.1 g tissue with 0.5 mL NS) by crushing for 10 min and then centrifuging at 3000 rpm for 10 min at 4° C. The 100 µL of supernatant of the lung homogenates was collected to extract viral RNA and qRT-PCR was used to assess the SARS-CoV-2 RNA copies in the infected lungs. All mice were euthanized after 4 day’s treatments, the body weight loss, SARS-CoV-2 RNA copies in the lungs, lung pathological changes, and key inflammatory cytokine levels (IL-6, IL-1β, TNF-α) in the lungs were studied.

### Pathological Examination of SARS-CoV-2 Infected and GA-Treated Mice

Experimental mice were anesthetized and the lungs were collected and fixed in 4% (v/v) paraformaldehyde solution for 48 hours, and paraffin sections (3-4 µm) were prepared. The paraffin sections were stained with Hematoxylin and Eosin (H&E) to identify histopathological changes in the lungs. The histopathology images of the lung tissues were observed by light microscopy. All experiments with SARS-CoV-2 were conducted in the Biosafety Level 3 (BSL3) Laboratories of the National Institute for Viral Disease Control & Prevention, Chinese Center for Disease Control and Prevention.

### Bio-Distribution, Side Effects, and Pharmacokinetics Study of GA Gold Compounds in Mice or Rats

BALB/c female mice and SD rats for experiments were conducted in compliance with regulations of the National Act on the use of experimental animals (China) and were approved by the Institutional Animal Care and Ethics Committee at the Chinese Academy of Sciences (approved No. SYXK (jing) 2014-0023). The experiment mice were intraperitoneally injected with GA at a dose of 15 mg/kg for 4 times (once a day for 4 days). Six hours after the last GA injection, the mice were anesthetized, and half of the organ tissues were analyzed by ICP-MASS to determine the distributions of Au atoms in blood, brain, heart, lung, liver, spleen, and kidney tissues. The other half of the organ tissues were fixed in 4% (v/v) paraformaldehyde solution for 48 hours, and paraffin sections (3-4 µm) were prepared. The paraffin sections were stained with Hematoxylin and Eosin (H&E) to identify histopathological changes. The Au content in the various organ tissues was measured with ICP-MS (Thermo-X7). For male and female SD rats, after intraperitoneal injection or intravenous injection of 5 mg Au/kg.bw, respectively, blood were collected from the jugular vein at different time points. The blood level of Au was analyzed with ICP-MS (Thermo-X7). PK parameters were determined using a noncompartmental analysis with PKSolver.

Referring to FIG. 13, Panel (a) shows the time scheme of administering GA or NS in relation to the infection of mice with SARS-CoV-2 virus intranasally. The COVID-19 mice model was successfully generated. Panels (b) to (d) show the comparative results from the SARS-COV-2 infected mice treated by NS and GA. There was more body weight loss (Panel (b)), higher SARS-CoV-2 RNA copies in the lung (Panel (c)) and significantly more severe bronchopneumonia and interstitial pneumonia and infiltration of lymphocytes within the alveolar were observed (Panel (d)). The pathological scores of mice lung tissue were assessed by grading the injury from 0 to 4 in accordance with the INHAND scoring standard, the average pathological score of virus-infected mice treated by NS is about 3. However, the body weight loss of GA treated mice was less compared with that of NS-treated COVID-19 mice. The number of viral RNA copies in the lungs of GA-treated mice was about 4×log10⁴, significantly lower than that in the lungs of NS-treated infected mice in which the number of viral RNA copies was about 5×log10⁵.

Referring to FIG. 14, histopathological analyses of the lung tissues of the GA treated COVID-19 mice in comparison with NS treated COVID-19 mice are shown. The SARS-CoV-2 infected mice treated with NS showed severe lung inflammation. The alveolar septum, bronchus, bronchioles, and perivascular interstitium were significantly widened, along with more lymphocytes and a small number of neutrophils infiltration. Also, a small number of lymphocytes and exfoliated epithelial cells were found in the lumen of local bronchioles of NS treated mice. However, GA-treated mice abrogated the characteristic signs of lung inflammation in SARS-CoV-2 infected mice. Local alveolar septum, bronchi, bronchiole and perivascular interstitial-widening significantly decreased. Although there was still some lymphocytic infiltration, the mucosal epithelium of bronchus and bronchioles were intact, and there were no foreign cells in the lumen, which showed a comparable level to those lung tissue sections of the mock mice that were not infected by the SARS-CoV-2. The mean pathological score obtained from histopathological lung observation further demonstrated that GA gold cluster significantly (p < 0.001) reduced pathological scores (about 1.8) compared with those of SARS-CoV-2 infected mice treated with NS (about 3.0).

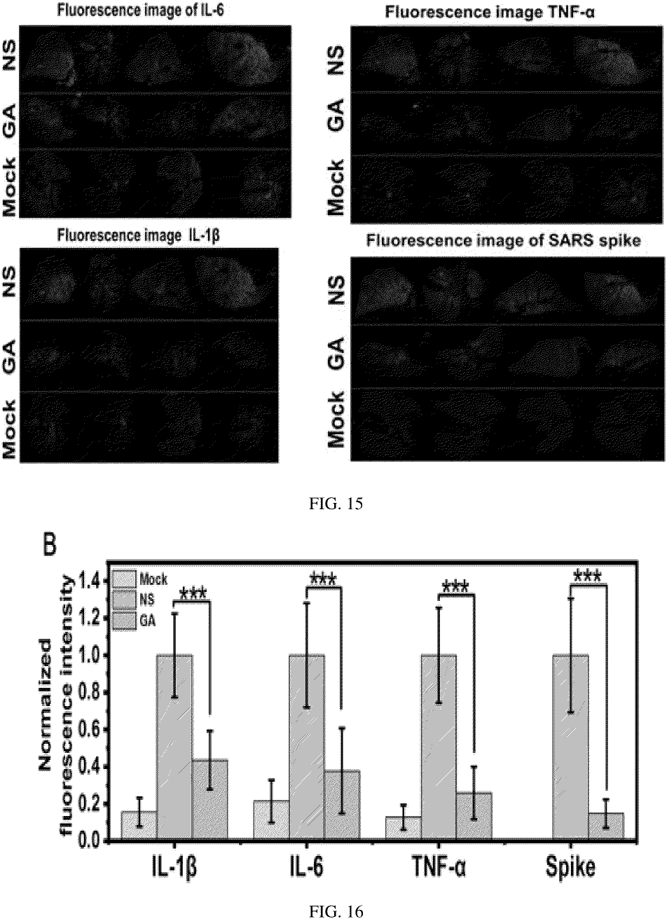

Referring to FIG. 15, the levels of the inflammatory cytokines in the lungs of COVID-19 mice were shown by immuno-fluorescence imaging. The levels of IL-6, IL-1β, TNF-α in the lungs of GA-treated COVID-19 mice were visibly lower than those of NS-treated COVID-19 mice. The GA treatment of COVID-19 mice thus significantly protected the lungs from injury by both inhibiting virus replication and suppressing the inflammatory cytokine expression in SARS-CoV-2 infected mice. FIG. 16 quantitatively shows the levels of IL-6, IL-1β, TNF-α in the lungs of GA and NS-treated COVID-19 mice. The levels of cytokines were significantly lower in GA-treated COVID-19 mice.

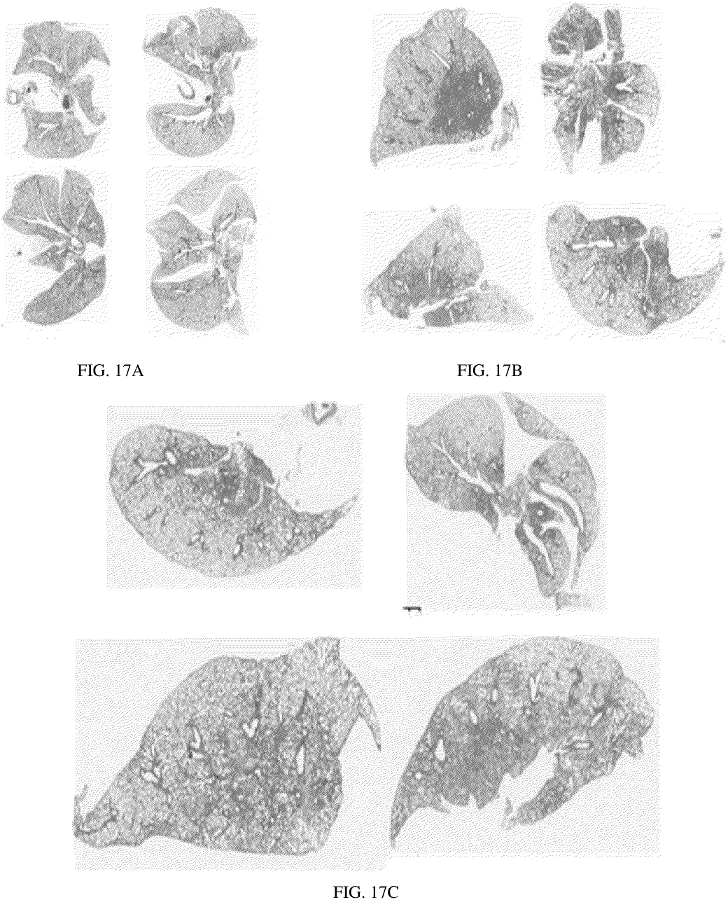

Referring to FIGS. 17A, 17B, and 17C, the pathological images of the lungs from normal mice, the NS-treated COVID-19 mice and the GA-treated COVID-19 mice are shown. The SARS-CoV-2 infected mice treated with NS showed severe lung inflammation (FIG. 17B). The alveolar septum, bronchus, bronchioles, and perivascular interstitium showed lymphocytes and neutrophils infiltration. Also, a small number of lymphocytes and exfoliated epithelial cells were found in the lumen of local bronchioles of NS-treated mice. It appears that treatment with GA for four days abrogated the characteristic signs of lung inflammation in SARS-CoV-2 infected mice (FIG. 17C). Local alveolar septum, bronchi, bronchiole, and perivascular interstitial widening were significantly decreased. There was much less lymphocytic infiltration, and the mucosal epithelium of the bronchus and bronchioles were intact, no foreign body were found in the lumen, and the lumen images were in comparable shape to those lung tissue sections of those normal mice that were not infected by the SARS-CoV-2 (FIG. 17A).

### The Bio-Distribution, Tissue Pathologic, and Pharmacokinetic Studies of GA in Mice/Rat

To check the tissue distribution of the Au ingredient and see if the Au ingredient induced tissue side effects, six normal BLAC/C mice in the treatment group were intraperitoneally injected with 15 mg/kg.bw GA 4 times for 1 time/day, and the mice in the control group were injected with NS in the same way. During this study, no side effects in the GA-treated mice were observed on aspects of movement, out-looking, sleeping, and eating behaviors.

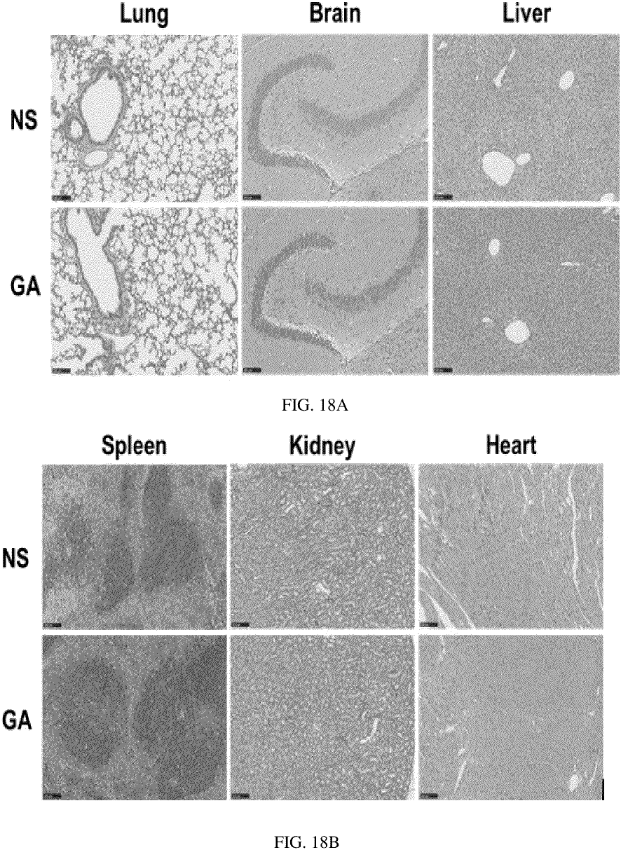

In reference to FIG. 18A and FIG. 18B, the pathological images of mouse brains, hearts, livers, lungs, spleens, and kidneys were shown after treating normal mice with GA for four days. Mouse tissue sections were dyed with Hematoxylin-eosin (HE). No statistically significant pathological changes were found in these tissues from the GA-treated normal mice compared with those of NS-treated normal mice, which suggests that 15 mg/kg.bw GA treatment to the mice should be safe for the mice in this study.

The Au ingredient distribution in mice organs was analyzed by ICPMASS and the results are shown in Table III. In the lung, the Au element concentration is about 51.07 µg/g, which may account for the GA-related inhibition of virus replication and the suppression of inflammatory cytokine expression. The gold distribution in hearts, livers, kidneys, brains, and spleens can be beneficial for COVID-19 treatment as they may potentially inhibit SARS-CoV-2 replication and suppress the inflammation cytokine level in those organs. As shown in Table III, the Au ingredient is mainly concentrated in the spleen, the heart, and the kidney. The high level of Au in kidney implied the Au ingredient may quickly excrete via urine, which is consistent with the pharmacokinetics data of GA in the rat model shown in Table IV.

The pharmacokinetics parameters of GA gold cluster via intraperitoneal injection of rats were Table IV. After rats were intraperitoneally injected with 5 mg/kg.bw of GA for one time, Au concentrations in the plasma were tested at different time points and kinetic characteristics of the gold cluster in rats were analyzed. According to the calculated parameters, the values of Tₘₐₓ for GA in male or female rats were 2 hours and the values of Cₘₐₓ for GA in male or female rats were 29.99 µg/mL or 31.750 µg/mL, respectively. The values of t1/2z for GA in male or female rats were 21.626 hr or 11.068 hr, respectively. Combine the data analysis of intravenous injection of GA at 5 mg/kg.bw, the F values of bioavailability for GA in male or female rats were 92.06% or 96.41%, respectively. These data confirm that GA has a favorable in vivo bioavailability in terms of pharmacological values.

TABLE III

Au distribution in mice tissues

| Tissue | Concentration (µg/g) |
|---|---|
| plasma | 15.01±0.30 |
| brain | 2.52±0.90 |
| heart | 10.35± 3.01 |
| lung | 51.70± 9.90 |
| spleen | 294.72± 12.35 |
| liver | 312.65±7.42 |
| kidney | 623.64±22.66 |

TABLE IV

Pharmacokinetics of intraperitoneal injection GA in SD rats

| parameter | unit | value | |
|---|---|---|---|
| | | Female | Male |
| AUC₍₀₋ₜ₎ | mg/L^{∗}h | 533.680 | 509.615 |
| AUC_{(0-∞)} | mg/L^{∗}h | 599.607 | 785.906 |
| AµMC₍₀₋ₜ₎ | | 6371.497 | 7609.285 |
| AµMC_{(0-∞)} | | 9797.793 | 26177.773 |
| MRT₍₀₋ₜ₎ | h | 11.939 | 14.931 |
| MRT_{(0-∞)} | h | 16.340 | 33.309 |
| VRT₍₀₋ₜ₎ | h^2 | 83.183 | 123.090 |
| VRT_{(0-∞)} | h^2 | 258.916 | 1045.121 |
| t_{1/2z} | h | 11.068 | 21.626 |
| Tₘₐₓ | h | 2 | 2 |
| CL_{Z}/F | L/h/kg | 0.008 | 0.006 |
| V_{Z}/F | L/kg | 0.133 | 0.199 |
| Cₘₐₓ | mg/L | 31.750 | 29.990 |
| F | % | 96.407 | 92.060 |

### GA Treatment of COVID-19 Golden Hamster Rat Via Intranasal Administration

The COVID-19 golden hamster rat model was generated following recently reported methods and detailed procedures. See Sin FunSia, et al, “Generation of a broadly useful model for COVID-19 pathogenesis, vaccination, and treatment,” Nature 583, 834-838 (2020). In particular, pathogen-free golden hamster rats were purchased from SiPeiFu Laboratory Animal Co (Beijing, China). All protocols were approved by the Institutional Animal Care and Use Committees of the National, Institute for Viral Disease Control & Prevention, the Chinese Center for Disease Control and Prevention. The SARS-CoV-2 strains used in this research were isolated from COVID-19 patients (BetaCoV/Wuhan/IVDC/-HB -01/2020, EPI_ISL_402119) and passaged on in Vero cells.

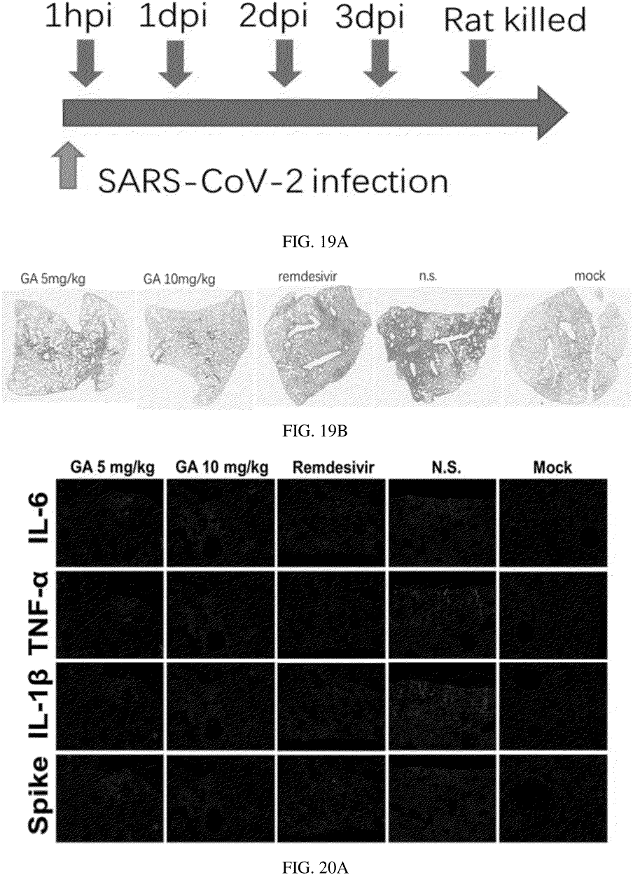

REMDESIBIR is an approved drug to treat COVID-19 in clinical. In this study, GA treatment is compared with REMDESIBIR treatment to determine the comparative drug efficiency for COVID-19 treatment. Similar to that described above, the golden hamster rats were divided into five groups. Rats were then infected intranasally using SARS-CoV-2 (1×10⁵ PFU) in a total volume of 50 µL DMEM. One hour after SARS-CoV-2 infection, rats in the NS group received normal saline (0.9% NaCl) via intranasal spray, rats in the REMDESIBIR group received a dose of 25 mg/kg.bw via intranasal spray, and rats in GA5mg/kg groups and GA10mg/kg groups received respectively a dose of GA at 5 mg Au/kg.bw and 10 mg Au/kg.bw via intranasal spray. Rats in the Mock group are not infected by SARS-Cov-2 and were used as control. Referring to FIG. 19A, the treatments follow the following process: various rat groups were infected with a virus on day 0, the rat groups were administered through an intranasal spray of doses of either GA or REMDESIBIR or normal saline treatment daily as shown in FIG. 19A for 3 days. All experiment rats were euthanized on day 4, and their pathological changes in lung tissues, level of SARS-CoV-2 spikes in lungs, and levels of key inflammatory cytokines (IL-6, IL-1β, TNF-α) in lungs were subsequently measured.

The pathological changes in the rat lung tissues were used as key indicators to examine the therapeutic effects of GA agents and REMDESIBIR. The lungs of the experiment rats were evaluated by scoring the injuries in accordance with the International Harmonization of Nomenclature and Diagnostic Criteria (INHAND) scoring standard. As shown in FIG. 19B, the average pathological score of virus-infected rats in NS-treated group was approx. 3, where the alveolar septum, bronchus, and perivascular interstitium were shown to have significantly widened, there were also infiltrations of lymphocytes and neutrophils, typical symptoms of COVID-19. COVID-19 rats by nasal spray treatment of REMDESIBIR at the dose of 25 mg/kg.bw demonstrated an average pathological score of approx. 2.7, and COVID-19 rats by nasal spray treatment of GA at dose of 5 mg/kg.bw demonstrated an average pathological score of approx. 2.5. The treated COVID-19 rates of 5 mg/kg.bw GA and 25 mg/kg.bw REMDESIBIR still had visibly widened alveolar septum, bronchus, and perivascular interstitium, as well as a small number of infiltrations of lymphocytes and neutrophils. However, COVID-19 rats treated by the nasal spray treatment of GA at a dose of 10 mg/kg.bw showed a significantly lower level of lung inflammation. The local alveolar septum, bronchi, and perivascular interstitial were significantly less widened, the infiltration of lymphocytes and neutrophils were also in a smaller number, and the average pathological score was approx. 2.3. Therefore GA demonstrates a dose-dependent manner in improving the pathological injury scores of COVID-19 rats caused by SARS-CoV-2 inflammation. GA at a dose of 10 mg/kg.bw demonstrated a better therapeutic outcome than REMDESIBIR of a dose of 25 mg/kg.bw.

The levels of SARS-CoV-2 spikes and inflammatory cytokines in the lungs of the experiment rats were measured using immuno-fluorescent imaging. Shown in FIG. 20A and FIG. 20B are the SARS-CoV-2 spike expression levels in rats treated with GA of 5 mg/kg.bw, GA of 10 mg/kg.bw and REMDESIBIR were significantly (p < 0.005) lower than those found in the NS-treated group. The SARS-CoV-2 spike level in the lungs of the rats in the Remdesivir group was significantly (p < 0.005) higher than that in the GA5 mg/kg-treated group and the GA10 mg/kg-treated group. For inflammatory cytokine levels in the lungs of virus-infected rats, IL-6, IL-1β, and TNF-α of Remdesivir or GA-treated rats were significantly (p < 0.005) lower than those found in the NS-treated rats. But the IL-6, IL-1β, and TNF-α in the lungs of the rats in the Remdesivir group were statistically significantly (p < 0.005) higher than those of GA5 mg/kg and GA10mg/kg-treated group. Together, these results demonstrated that both drugs were effect through intranasal administration. Both demonstrated also inhibition of virus replication and reduced levels of cytokine expression in the infected lungs. The GA shows higher efficacy in the inhibition of virus replication and in the suppression of inflammatory cytokine expression level compared with Remdesivir in COVID-19 golden hamster rat model.

### The Effect of a Second Gold Cluster Compound Au₂₈(SV)₉ on the Inhibition of SARS-CoV-2 M^{pro} Activity

Au₂₅(SV)₉ was obtained by using SV peptide CysCysTyrGlyGlyProLysLysLysArgLysProGly (SEQ ID NO. 6).

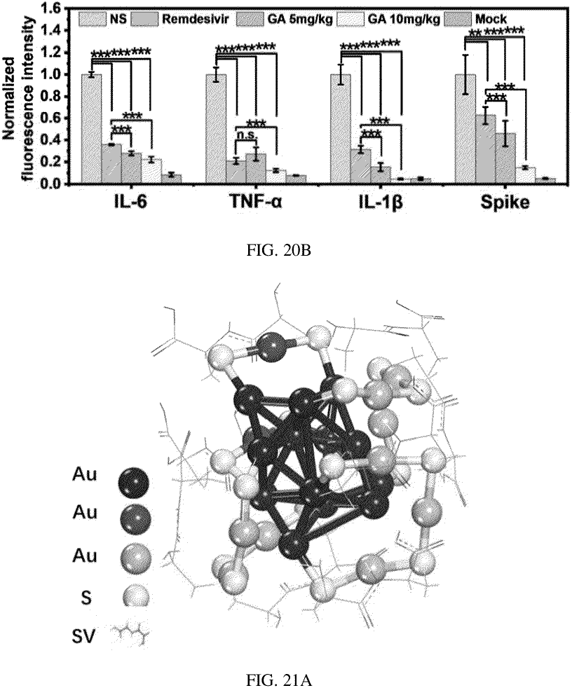

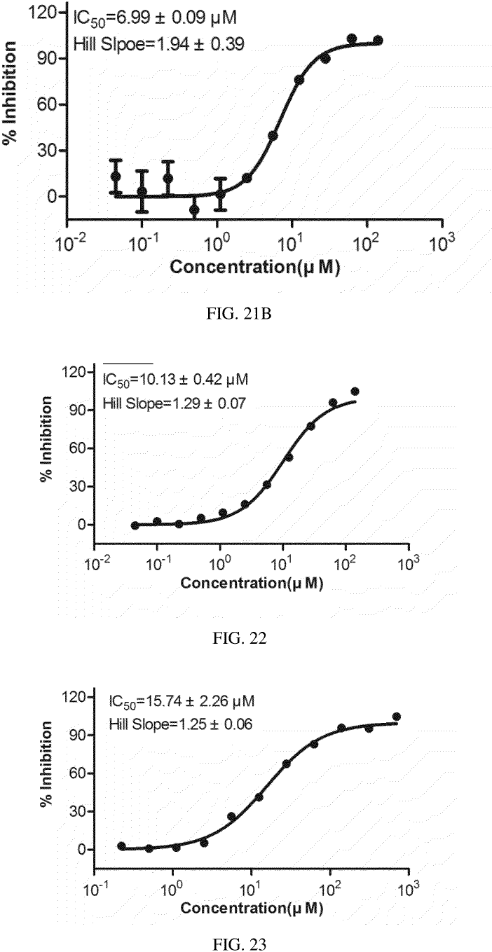

FIG. 21A shows the molecule structure of Au₂₅SV₉. FIG. 21B shows that the 50%inhibition of purified M^{pro} protease activity (IC50) reached about 6.9 µM Au₂₈SV₉.

FIG. 22 shows the suppression of M^{pro} protease activity by Au₁₀(SG)₁₀.

FIG. 23 shows the suppression of M^{pro} protease activity by Au₄₃(SG)₃₇.

This application describes a therapeutic composition for treatment of coronavirus infection in an animal, comprising: a gold compound stored in a liquid form as an active ingredient wherein a gold atom forms an Au—S bond in the coronavirus’ protease active pocket.

According to one embodiment of the therapeutic composition for treatment of coronavirus infection in the present application, the gold compound is AURANOFIN having molecule formula C₂₀H₃₄AuO₉PS.

According to one embodiment of the therapeutic composition for treatment of coronavirus infection in the present application, the gold compound is Aurothioglucose having molecule formulaC₆H₁₁AuO₅S.

According to one embodiment of the therapeutic composition for treatment of coronavirus infection in the present application, the gold compound is aurothiomalate having molecule formula C₄H₅AᵤO₄S.

According to one embodiment of the therapeutic composition for treatment of coronavirus infection in the present application, the gold compound is a gold cluster complex, wherein multiple gold atoms and multiple peptides or proteins or polymers form a complex molecule.

According to one embodiment of the therapeutic composition for treatment of coronavirus infection in the present application, the gold compound has a chemical formula Auₘ(SG)ₙ wherein Au represents gold atoms, SG represents a small peptide, m is an integer between 2-200 and n is an integer between 3-202.

According to one embodiment of the therapeutic composition for treatment of coronavirus infection in the present application, SG represents a glutathione peptide (SEQ ID NO. 1) or SV peptide (SEQ ID NO. 6).

According to one embodiment of the therapeutic composition for treatment of coronavirus infection in the present application, the gold compound are Au₁₀₋₁₂(SG)₁₀₋₁₂, Au₁₅(SG)₁₃, Au₁₈(SG)₁₄, Au₂₂(SG)₁₆, Au₂₂(SG)₁₇, Au₂₅(SG)₁₈, Au₂₅(SG)₉, Au₂₉(SG)₂₀, Au₂₉(SG)₂₇, Au₃₀(SG)₂₈,Au₃₃(SG)₂₂, Au₃₅(SG)₂₂, Au₃₈(SG)₂₄, Au₃₉(SG)₂₄, Au₁₈(SG)₁₁, Au₂₁(SG)₁₂, Au₂₅(SG)₁₄, Au₂₈(SG)₁₆, Au₃₂(SG)₁₈, and Au₃₉(SG)₂₃, Au₄₃(SG)₃₇ wherein SG represents a glutathione peptide (SEQ ID NO. 1) or SV (SEQ ID NO. 6) peptide.

According to one embodiment of the therapeutic composition for treatment of coronavirus infection in the present application, the gold compound is a gold cluster complex having a molecular formula Au₂₉(SG)₂₇ wherein SG represents a glutathione peptide (SEQ ID NO. 1) that Au represents a gold atom

According to one embodiment of the therapeutic composition for treatment of coronavirus infection in the present application, the coronavirus infection is COVID-19 and the coronavirus is SARS-CoV-2.

This application also describes a method for treating a coronavirus infection in an animal, said method comprising steps of: preparing a therapeutic composition of Claim 1, and administering a sufficient amount of said therapeutic composition to said animal.

According to one embodiment of the method for treating a coronavirus infection in the present application, the step of administering is carried out through an intranasal spray method.

According to one embodiment of the method for treating a coronavirus infection in the present application, said step of administering is carried out through intraperitoneal injection or intramuscular injection, or an intravenous-injection method

According to one embodiment of the method for treating a coronavirus infection in the present application, the step of administering is carried out through a nasal inhaling method.

According to one embodiment of the method for treating a coronavirus infection in the present application, the step of preparing a therapeutic agent further comprises the step of reacting gold (I) or gold (III) salt with glutathione peptide (SEQ ID NO. 1) solutions

According to one embodiment of the method for treating a coronavirus infection in the present application, the sufficient amount is in the range of 1 mg/kg.bw to 30 mg/kg.bw of the animal.

This application describes a therapeutic composition for treatment of coronavirus infection in an animal, the coronavirus encoding a conserved papain-like main protease critical for its replication, comprising: a gold cluster compound stored in a liquid form having a molecular formula chemical Auₘ(SG)ₙ wherein Au represents gold atoms, SG represents a small peptide, m is an integer between 2-200 and n is an integer between 3-202 wherein SG represents glutathione peptide (SEQ ID NO. 1) or SV peptide (SEQ ID NO. 6) wherein said gold cluster compound functions as an active ingredient that both forms Au—S bond in the active pocket of the main protease and suppresses cytokine expressions in the animal’s body.

According to one embodiment of the therapeutic composition for treatment of coronavirus infection in the present application, the gold cluster compound is administered to said animal for at least 4 days.

According to one embodiment of the therapeutic composition for treatment of coronavirus infection in the present application, the gold cluster compound is administered to said animal through a nasal spray method.

According to one embodiment of the therapeutic composition for treatment of coronavirus infection in the present application, the gold cluster compound is administered to said animal through transdermal absorption.

As will be recognized by those skilled in the art, the innovative concepts described in the present application can be modified and varied over a tremendous range of applications, and accordingly, the scope of patented subject matter is not limited by any of the specific exemplary teachings given. It is intended to embrace all such alternatives, modifications, and variations that fall within the spirit and broad scope of the appended claims.

Additional general background, which helps to show variations and implementations, may be found in the following publications, all of which are hereby incorporated by reference herein for all purposes.

Under no circumstances the description in the present application should be read as implying that any particular element, step, or function is an essential element that must be included in the claim scope. The scope of patented subject matter is defined only by the allowed claims. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

1. A therapeutic composition for treatment of coronavirus infection in an animal, comprising:
a gold compound stored in a liquid form as an active ingredient, wherein a gold atom forms an Au—S bond in the coronavirus’ protease active pocket.

2. The therapeutic composition of claim 1, wherein the gold compound is AURANOFIN having molecule formula C₂₀H₃₄AuO₉PS, C₆H₁₁AuO₅S, or C₄H₅AuO₄S.

3. The therapeutic composition of claim 1, wherein the gold compound is a gold cluster complex wherein multiple gold atoms and multiple peptides or proteins or polymers form a complex molecule.

4. The therapeutic composition of claim 3, wherein the gold compound has a chemical formula Auₘ(SG)ₙ, wherein Au represents gold atoms, SG represents a small peptide, m is an integer between 3-200 and n is an integer between 2-220.

5. The therapeutic composition of claim 4, wherein m is an integer between 10-43 and n is an integer between 10-42.

6. The therapeutic composition of claim 4, wherein SG represents a glutathione peptide (SEQ ID NO. 1) or SV peptide (SEQ ID NO. 6).

7. The therapeutic composition of claim 6, wherein the gold compound is selected from group consisting of Au₁₀₋₁₂(SG)₁₀₋₁₂, Au₁₅(SG)₁₃, Au₁₈(SG)₁₄, Au₂₂(SG)₁₆, Au₂₂(SG)₁₇, Au₂₂(SG)₁₈, Au₂₅(SG)₁₈, Au₂₅(SG)₉, Au₂₉(SG)₂₀, Au₂₉(SG)₂₇, Au₃₀(SG)₂₈, Au₃₃(SG)₂₂, Au₃₅(SG)₂₂, Au₃₈(SG)₂₄, Au₃₉(SG)₂₄, Au₁₈(SG)₁₁, Au₂₁(SG)₁₂, Au₂₅(SG)₁₄, Au₂₈(SG)₁₆, Au₃₂(SG)₁₈, Au₃₉(SG)₂₃, and Au₄₃(SG)₃₇.

8. The therapeutic composition of claim 7, wherein the gold compound is a gold cluster complex having a molecular formula Au₂₉(SG)₂₇, wherein SG represents a glutathione peptide (SEQ ID NO. 1) and Au represents a gold atom.

9. The therapeutic composition of claim 1 wherein the coronavirus infection is COVID-19, the coronavirus is SARS-CoV-2, and the animal is human.

10. (canceled)

11. A method for treating or preventing coronavirus infection in an animal, wherein the method comprises a step of:
administering a sufficient amount of said therapeutic composition according to claim 1 to said animal.

12. The method of claim 11, wherein said step of administering is conducted through intranasal spray, intraperitoneal injection, intramuscular injection, intravenous-injection, or a nasal inhaling method.

13. The method of claim 11, wherein the method further comprises a step of preparing the therapeutic composition, and said step of preparing the therapeutic composition further comprises a step of reacting a gold (I) or gold (III) salt with glutathione peptide (SEQ ID NO. 1) solutions.

14. The method of claim 11, wherein the sufficient amount is in the range of 1 mg/kg.bw to 30 mg/kg.bw of the animal.

15. A therapeutic composition for treatment of coronavirus infection in an animal, the coronavirus encoding a conserved papain-like main protease critical for replication of the coronavirus, comprising:
a gold cluster compound stored in a liquid form having a molecular formula of Auₘ(SG)ₙ, wherein Au represents a gold atom, SG represents a small peptide, m is an integer between 2-200 and n is an integer between 3-202, wherein SG represents glutathione peptide (SEQ ID NO. 1) or SV peptide (SEQ ID NO. 6), and wherein said gold cluster compound functions as an active ingredient that both forms Au—S bond in the active pocket of the main protease and suppresses cytokine expression in body of an animal.

16. The therapeutic composition of claim 15, wherein the gold cluster compound is administered to said animal for at least 4 days.

17. The therapeutic composition of claim 15, wherein the gold cluster compound is administered to said animal through a nasal spray method.

18. The therapeutic composition of claim 15, wherein the gold cluster compound is administered to said animal through transdermal absorption.

19. A method for treating or preventing coronavirus infection in an animal, wherein the method comprises a step of administering a sufficient amount of said therapeutic composition according to claim 2 to said animal.

20. A method for treating or preventing coronavirus infection in an animal, wherein the method comprises a step of administering a sufficient amount of said therapeutic composition according to claim 3 to said animal.

21. A method for treating or preventing coronavirus infection in an animal, wherein the method comprises a step of administering a sufficient amount of said therapeutic composition according to claim 7 to said animal.


SEQUENCE LISTING (6 sequences)

SEQ ID NO: 1 - protein, 3 aa, Artificial Sequence
  Amino acid sequence of glutathion
ECG

SEQ ID NO: 2 - DNA, 25 bp, Artificial Sequence
  Forward primer targeting SARS-COV-2 ORF1a
agaagattggttagatgatgatagt

SEQ ID NO: 3 - DNA, 25 bp, Artificial Sequence
  Reverse primer targeting SARS-COV-2 ORF1a
ttccatctctaattgaggttgaacc

SEQ ID NO: 4 - DNA, 24 bp, Artificial Sequence
  Probe targeting SARS-COV-2 ORF1a
tcctcactgccgtcttgttgacca

SEQ ID NO: 5 - protein, 12 aa, Artificial Sequence
  Substrate for Mpro protease reaction
ESATLQSGLAKS

SEQ ID NO: 6 - protein, 13 aa, Artificial Sequence
  Synthetic small peptide
CCYGGPKKKRKPG